(19) United States
(12) Patent Application Publication
(10) Pub. No.: US 2019/0282508 A1
(43) Pub. Date: Sep. 19, 2019

(54) EXTENDED RELEASE DRUG FORMULATION WITH OVERDOSE PROTECTION AND ABUSE DETERRENCE

(71) Applicant: KASHIV PHARMA LLC, Bridgewater, NJ (US)

(72) Inventors: Navnit H. Shah, Clifton, NJ (US); Wantanee Phuapradit, Montville, NJ (US); Dipen Desai, Whippany, NJ (US); Kiran Siva Ram Vaka, Piscataway, NJ (US); Kanji Meghpara, Morris Plains, NJ (US); Atsawin Thongsukmak, Piscataway, NJ (US)

(73) Assignee: KASHIV PHARMA LLC, Bridgewater, NJ (US)

(21) Appl. No.: 16/074,506

(22) PCT Filed: Feb. 1, 2017
(86) PCT No.: PCT/US2017/016076

§ 371 Date: Aug. 1, 2018

Related U.S. Application Data
(60) Provisional application No. 62/289,733, filed on Feb. 1, 2016.
(60) Provisional application No. 62/331,285, filed on May 3, 2016.

(51) Int. Cl. A61K 9/28; A61K 9/50; A61K 47/64; A61K 47/10; A61K 47/14; A61K 31/485; A61K 9/20

(52) U.S. Cl. CPC A61K 9/2846; A61K 9/5021; A61K 9/5073; A61K 9/5084; A61K 47/645; A61K 9/2086; A61K 47/14; A61K 31/485; A61K 9/2866; A61K 9/2886; A61K 47/10

(57) ABSTRACT

The presently disclosed subject matter provides solid, oral, extended release, pharmaceutical particulate and multi-particulate dosage forms with abuse deterrent and overdose protection features/characteristics comprising at least one or two populations of particulates. In certain embodiments, the first population of particulates comprises a therapeutically effective amount of at least one opioid embedded in a polymer matrix, a primary functional coat layer (FC 1), a secondary functional coat 2 coat layer (FC 2), and an over coat; wherein FC 1 comprises a nonionic water-insoluble polymer and, optionally, at least one of a cationic polymer, a nonionic water-soluble polymer, and a water-soluble plasticizer; FC 2 comprises a cationic polymer and, optionally, a nonionic water-insoluble polymer; and the over coat comprises a nonionic water-soluble polymer. The second population of particulates comprises an alkaline agent and, optionally, a pH-stabilizing agent. In certain embodiments, the extended release pharmaceutical dosage form contains at least three different populations of multi-particulates. Each population of particulates is designed for a specific function to accomplish the desired combination of abuse deterrence and overdose protection. The presently disclosed subject matter also provides methods related to the solid, oral, extended release, particulate and multi-particulate dosage forms.

This application is a U.S. National Stage patent application under 35 U.S.C. § 371 of International Application No. PCT/US2017/016076, filed on Feb. 1, 2017, which claims the benefit of priority to U.S. Provisional Patent Application No. 62/289,733, filed Feb. 1, 2016, and U.S. Provisional Patent Application No. 62/331,285, filed May 3, 2016, the disclosures of each of which are hereby incorporated by reference herein in their entireties.

### 1. FIELD OF THE INVENTION

The present disclosure relates to tamper- and/or overdose-resistant extended release pharmaceutical dosage forms and processes of manufacture.

### 2. BACKGROUND

Governmental reports state that prescription drug abuse is the fastest growing drug problem in the United States, and a survey indicated that nearly one-third of people age 12 and above who used drugs illicitly for the first time in 2009 began by the nonmedical use of a prescription drug. For example, prescription opioid analgesics can be abused by swallowing whole in excessive quantities (multi-tablet dosing); crushing and swallowing; crushing and inhaling nasally or insufflating nasally (“snorting”); crushing and smoking; or crushing, dissolving, and injecting the prescription opioid.

Drug abuse often involves some physical manipulation of a dosage form so that a larger amount of the drug formulated in an extended release dosage form is available immediately to be taken orally, nasally, or by intravenous injection. In addition, people deliberately or mistakenly can swallow a number of intact pills or tablets despite instructions to the contrary, and they can suffer serious side effects. Products containing active ingredients that will produce an emotional, psychological, euphoric, or depressive experience are particularly vulnerable to this form of abuse. Hence, there is a need for abuse-deterrent opioid dosage forms that can also prevent, inhibit, or delay the adverse effects of an overdose caused by ingesting multiple units of the dosage form, either intentionally or unintentionally.

In March 2016, the FDA published a guidance document describing general procedures for developing and evaluating abuse deterrence of generic solid oral opioid products formulated to incorporate physical or chemical barriers, agonists/antagonists, aversive agents, or combinations of these technologies. The FDA recommends the following evaluations, involving all potential routes of abuse, of the abuse deterrence of generic solid oral opioid drug products:
1. Injection (parenteral route)—evaluate the extractability and syringeability of intact and mechanically manipulated products.
2. Ingestion (oral route)—evaluate extractability, dissolution, and where applicable, the rate and extent of a product's absorption for intact, and mechanically or chemically manipulated, products.
3. Insufflation (nasal route)—evaluate nasal availability and likability of mechanically manipulated and insufflated products.
4. Smoking (inhalation route)—evaluate the ability to sublimate intact, and mechanically or chemically manipulated, products.
   The FDA further describes mechanical manipulation, with and without thermal pretreatment (e.g., heating or freezing), as involving cutting, grating, and milling.

Oral drug administration remains the route of choice for the majority of clinical applications. Modified release (MR) dosage forms that are administered once or twice daily offer advantages over their immediate release (IR) counterparts because they reduce the magnitude of peaks and troughs of drug plasma concentration, and provide longer dosing intervals, sustained analgesic effect, and increased patient compliance. These modified release formulations can be referred to, for example, as extended release (ER), controlled release (CR), and/or sustained release (SR). For certain patients, such as those suffering from considerable pain, these MR products can reduce pain sufficiently to avoid the need for additional dosing. Thus, such formulations can significantly increase the quality of life for these patients. Both IR and MR products for pain are widely available in the market. Examples of IR products include those containing NSAIDs (e.g., naproxen), cox II inhibitors (e.g., celecoxib), and opioids (e.g., oxycodone). Examples of MR products include those containing NSAIDs and opioids (e.g., TYLENOL SR®, OXYCONTIN®).

A few abuse-resistant opioid products are currently approved for marketing, including OXYCONTIN® (oxycodone HCl extended release tablets), TARGINIQ® (oxycodone HCl and naloxone HCl), and EMBEDA® (morphine sulfate and naltrexone HCl). Other products such as SUBOXONE® (buprenorphine and naloxone) and OPANA ER® (oxymorphone HCl) also purport to have abuse-deterrent properties, but do not have a formal claim on the label. As noted by the FDA in their 2015 guidelines, most abuse-deterrent technologies have not yet proven successful at deterring the most common form of abuse: swallowing multiple intact capsules or tablets (multi-tablet dosing), in excess of the prescribed number of dosage form units.

A need, therefore, remains for improved formulations that make it difficult, if not impossible, for individuals to abuse or misuse opioids, not only by snorting and/or extraction of drug, but also by ingesting multiple doses. In particular, new formulations are needed that can be administered as IR and ER pharmaceutical products. In particular, new formulations are needed that can provide abuse deterrence, particularly overdose protection, to ER pharmaceutical products, which contain higher amounts of opioid in the dosage form. There is also a need for improved formulations that reduce or prevent the effects of overdose, whether intentional or unintentional (e.g., accidental overdose while legitimately seeking pain relief). Such formulations ideally would combine overdose protection and abuse deterrence in a single dosage form and thereby address multiple health-related concerns, especially regarding habit-forming opioid compounds for which there exists a high propensity for abuse and overdose. These dosage forms, while providing overdose protection by reducing or preventing the release of opioids, must also allow the active pharmaceutical ingredient to be released and solubilized in the gastrointestinal tract and have the desired pharmacological activity (e.g., an analgesic effect), when taken in prescribed amounts at prescribed dosing intervals.

### 3. SUMMARY OF THE INVENTION

The present disclosure provides a solid oral extended release multi-particulate dosage form with abuse deterrent and overdose protection characteristics comprising (a) a first population of particulates comprising a therapeutically effective amount of at least one opioid embedded in a polymer matrix, a primary functional coat layer (FC 1) over the polymer matrix, a secondary functional coat layer (FC 2) over FC 1, and an over coat over FC 2, wherein FC 1 comprises a nonionic water-insoluble polymer and, optionally, at least one of a cationic polymer, a nonionic water-soluble polymer, and a water-soluble plasticizer; wherein FC 2 comprises at least one of a cationic polymer, a nonionic water-soluble polymer, and a water-soluble plasticizer and, optionally, a nonionic water-insoluble polymer; and wherein the over coat comprises a nonionic water-soluble polymer; and (b) a second population of particulates comprising an alkaline agent and, optionally, a pH-stabilizing agent, wherein the dosage form is suitable for twice-daily administration and provides an extended release of the opioid for a period of at least about 4 hours, and releases less than about 40% by weight of the opioid or a pharmaceutically acceptable salt thereof from the dosage form at about 1 hour; and wherein, when two or more dosage units are consumed, the alkaline agent raises the gastric pH and the pH-stabilizing agent, when present, maintains the elevated pH to further extend the release of the opioid from the dosage form.

The present disclosure also provides a solid oral extended release multi-particulate dosage form with abuse deterrent and overdose protection characteristics comprising: (a) a first population of particulates comprising a therapeutically effective amount of at least one opioid embedded in a polymer matrix, a primary functional coat layer (FC 1) over the polymer matrix, a secondary functional coat layer (FC 2) over FC 1, and an over coat over FC 2, wherein FC 1 comprises a nonionic water-insoluble polymer and, optionally, at least one of a cationic polymer, a nonionic water-soluble polymer, and a water-soluble plasticizer; wherein FC 2 comprises at least one of a cationic polymer, a nonionic water-soluble polymer, and a water-soluble plasticizer and, optionally, a nonionic water-insoluble polymer; and wherein the over coat comprises a nonionic water-soluble polymer; and (b) a second population of particulates comprising an alkaline agent and a pH-stabilizing agent, wherein the dosage form is suitable for once-daily administration and provides an extended release of the opioid for a period of at least about 8 hours, and releases less than about 40% by weight of the opioid or a pharmaceutically acceptable salt thereof from the dosage form at about 1 hour; and wherein, when two or more dosage units are consumed, the alkaline agent raises the gastric pH and the pH-stabilizing agent maintains the elevated pH to further extend the release of the opioid from the dosage form.

In certain embodiments, the present disclosure provides a dosage form where at least one cationic polymer, water-soluble plasticizer, and/or nonionic water-soluble polymer acts as a pore former in FC 1 at a nonionic water-insoluble polymer to cationic polymer, water-soluble plasticizer, and/or nonionic water-soluble polymer ratio of from about 80:20 to about 99.9:0.1 wt % ratio.

In certain embodiments, the present disclosure provides a dosage form where the wt % ratio of the nonionic water-insoluble polymer to the cationic polymer in FC 1 is about 95:5.

In certain embodiments, the present disclosure provides a dosage form where the wt % ratio of the nonionic water-insoluble polymer to the cationic polymer in FC 1 is about 98:2.

In certain embodiments, the present disclosure provides a dosage form where the wt % ratio of the nonionic water-insoluble polymer to the nonionic water-soluble polymer in FC 1 is about 95:5.

In certain embodiments, the present disclosure provides a dosage form where FC 2 comprises a cationic polymer and a water-soluble plasticizer.

In certain embodiments, the present disclosure provides a dosage form where the nonionic water-insoluble polymer is selected from the group consisting of cellulose acetate, cellulose acetate-based polymers, ethylcellulose, and polyvinyl acetate polymers.

In certain embodiments, the present disclosure provides a dosage form where the nonionic water-insoluble polymer is cellulose acetate.

In certain embodiments, the present disclosure provides a dosage form where the nonionic water-soluble polymer is hydroxypropyl methylcellulose (HPMC).

In certain embodiments, the present disclosure provides a dosage form where the water-soluble plasticizer is triethyl citrate and/or a polyethylene glycol (MW 400 8000).

In certain embodiments, the present disclosure provides a dosage form where the cationic polymer present in FC 2 and, optionally, in FC 1, is a copolymer based on dimethylaminoethyl methacrylate, butyl methacrylate, and methyl methacrylate.

In certain embodiments, the present disclosure provides a dosage form where the polymer matrix comprises a nonionic pH-independent polymer.

In certain embodiments, the present disclosure provides a dosage form where the polymer matrix comprises a nonionic pH-independent polymer and an anionic pH-dependent polymer.

In certain embodiments, the present disclosure provides a dosage form where the anionic pH-dependent polymer is a carbomer.

In certain embodiments, the present disclosure provides a dosage form where the carbomer provides resistance to extraction of the opioid from the dosage form into a dissolution medium or gastrointestinal (GI) fluid, and provides resistance to extraction of the opioid into a syringe when two or more dosage units are taken together or dissolved in the dissolution medium.

In certain embodiments, the present disclosure provides a dosage form where the dissolution medium comprises aqueous and/or hydro-organic solvents.

In certain embodiments, the present disclosure provides a dosage form where the nonionic pH-independent polymer is selected from the group consisting of a copolymer of ethyl acrylate, methyl methacrylate, and a low content of methacrylic acid ester with quaternary ammonium groups (ammonium methacrylate copolymer), hydroxypropylcellulose, HPMC, hydroxyethylcellulose, ethylcellulose, cellulose acetate butyrate, cellulose acetate, polyvinyl acetate polymers, and polyethylene oxide polymers.

In certain embodiments, the present disclosure provides a dosage form where the nonionic pH-independent polymer is a polyethylene oxide polymer and/or HPMC, or a polyvinyl acetate-polyvinyl pyrrolidone polymer.

In certain embodiments, the present disclosure provides a dosage form where the polyethylene oxide polymer provides resistance to extraction of the opioid from the dosage form into a dissolution medium or GI fluid, and provides resistance to extraction of the opioid into a syringe when two or more dosage units are dissolved in the dissolution medium or taken together.

In certain embodiments, the present disclosure provides a dosage form where the nonionic pH-independent polymer is a mixture of a polyethylene oxide polymer and HPMC.

In certain embodiments, the present disclosure provides a dosage form where the nonionic pH-independent polymer in the over coat comprises a cellulose ether polymer.

In certain embodiments, the present disclosure provides a dosage form where the cellulose ether polymer is HPMC.

In certain embodiments, the present disclosure provides a dosage form where the alkaline agent present in the second population of particulates is selected from the group consisting of aluminum hydroxide, sodium hydroxide, potassium hydroxide, calcium hydroxide, magnesium hydroxide, calcium carbonate, sodium carbonate, potassium bicarbonate, sodium bicarbonate, ammonia, tertiary sodium phosphate, diethanolamine, ethylenediamine, N methylglucamine, L lysine, and combinations thereof.

In certain embodiments, the present disclosure provides a dosage form where the alkaline agent is magnesium hydroxide.

In certain embodiments, the present disclosure provides a dosage form where the alkaline agent elevates the gastric pH to a value of greater than about 5 when two or more dosage units are taken together.

In certain embodiments, the present disclosure provides a dosage form where the pH-stabilizing agent present in the second population of particulates is dibasic calcium phosphate and/or tricalcium phosphate.

In certain embodiments, the present disclosure provides a dosage form where the polymer matrix further comprises an antioxidant, a plasticizer, and/or a surfactant.

In certain embodiments, the present disclosure provides a dosage form where the first population of particulates further comprises a seal coat disposed between the polymer matrix and FC 1.

In certain embodiments, the present disclosure provides a dosage form where the seal coat comprises a nonionic water-soluble polymer.

In certain embodiments, the present disclosure provides a dosage form where the nonionic water-soluble polymer comprises a cellulose ether polymer.

In certain embodiments, the present disclosure provides a dosage form where the cellulose ether polymer is HPMC.

In certain embodiments, the present disclosure provides a dosage form where the over coat is the outermost coat of the first population of particulates.

In certain embodiments, the present disclosure provides a dosage form where the first population of particulates further comprises at least one additional coating layer between the seal coat and FC1, or between FC 1 and FC 2, or between FC 2 and the over coat.

In certain embodiments, the present disclosure provides a dosage form where the opioid is selected from the group consisting of oxycodone, oxymorphone, hydromorphone, hydrocodone, buprenorphine, codeine, phenazocine, tilidine, tramadol, meperidine, sufentanil, prodine, methadone, pentazoxine, tapentadol, morphine, fentanyl, pharmaceutically acceptable salts thereof, and a mixture thereof.

In certain embodiments, the present disclosure provides a dosage form where the opioid is selected from the group consisting of oxycodone, hydrocodone, hydromorphone, oxymorphone, pharmaceutically acceptable salts thereof, and a mixture thereof.

In certain embodiments, the present disclosure provides a dosage form that further comprises a third population of particulates comprising a viscosity enhancing agent.

In certain embodiments, the present disclosure provides a dosage form where the viscosity enhancing agent is a viscosity-building polymer.

In certain embodiments, the present disclosure provides a dosage form where the viscosity-building polymer(s) is a nonionic polymer and/or an anionic polymer.

In certain embodiments, the present disclosure provides a dosage form where the nonionic polymer is a polyethylene oxide polymer and the anionic polymer is a carbomer.

In certain embodiments, the present disclosure provides a dosage form where the first population of particulates is present in an amount from about 10% to about 80% w/w of the total dosage form.

In certain embodiments, the present disclosure provides a dosage form where the second population of particulates is present in an amount from about 20% to about 42% w/w of the total dosage form.

In certain embodiments, the present disclosure provides a dosage form where the third population of particulates is present in an amount from about 2% to about 50% w/w of the total dosage form.

In certain embodiments, the present disclosure provides a dosage form where the abuse deterrent characteristics comprise syringeability resistance, extractability resistance in aqueous and/or hydro-organic solvents, resistance to alcohol dose dumping, and heat stability of the dosage form, wherein the heat stability comprises maintaining the abuse deterrent characteristics of the dosage form after the exposure to heat.

In certain embodiments, the present disclosure provides a dosage form where heat stability is determined by subjecting the dosage form to heating at about 100° C. for at least 2 hours in an oven, or heating in a microwave at 1200 W for at least 13-15 minutes.

In certain embodiments, the present disclosure provides a dosage form where the abuse deterrent characteristics comprise resistance to crushing and grinding of the first population of particulates.

In certain embodiments, the present disclosure provides a dosage form where the abuse deterrent characteristics comprise resistance to segregation of the opioid into the fines fraction of the dosage form upon grinding.

In certain embodiments, the present disclosure provides a dosage form where the fines fraction comprises a fraction of particulates with a size that can be snorted or insufflated.

The present disclosure also provides a method of preparing a solid oral extended release multi-particulate dosage form with abuse deterrent and overdose protection characteristics comprising: (a) preparing a first population of particulates comprising a therapeutically effective amount of at least one opioid embedded in a polymer matrix, a primary functional coat layer (FC 1) over the polymer matrix, a secondary functional coat layer (FC 2) over FC 1, and an over coat over FC 2, wherein FC 1 comprises a nonionic water-insoluble polymer and, optionally, at least one of a cationic polymer, a nonionic water-soluble polymer, and a water-soluble plasticizer; wherein FC 2 comprises at least one of a cationic polymer, a nonionic water-soluble polymer, and a water-soluble plasticizer and, optionally, a nonionic water-insoluble polymer; and wherein the over coat comprises a nonionic water-soluble polymer; (b) preparing a second population of particulates comprising an alkaline agent and a pH-stabilizing agent; and (c) combining the first and second populations of particulates; wherein the dosage form provides an extended release of the opioid for a period of at least about 4 hours, and releases less than about 40% by weight of the opioid or a pharmaceutically acceptable salt thereof from the dosage form at about 1 hour; and wherein, when two or more dosage units are consumed, the alkaline agent raises the gastric pH and the pH-stabilizing agent maintains the elevated pH to further extend the release of the opioid from the dosage form.

In certain embodiments, the present disclosure provides a method of preparing a dosage form where at least one cationic polymer, water-soluble plasticizer, and/or nonionic water-soluble polymer acts as a pore former in FC 1 at a nonionic water-insoluble polymer to cationic polymer, water-soluble plasticizer, and/or nonionic water-soluble polymer ratio of from about 80:20 to about 99.9:0.1 wt % ratio.

In certain embodiments, the present disclosure provides a method of preparing a dosage form further comprising coating the polymer matrix of the first population of particulates with a seal coat prior to coating with FC 1.

In certain embodiments, the present disclosure provides a method of preparing a dosage form where the seal coat comprises a nonionic water-soluble polymer.

In certain embodiments, the present disclosure provides a method of preparing a dosage form where FC 2 comprises a cationic polymer and a water-soluble plasticizer.

In certain embodiments, the present disclosure provides a method of preparing a dosage form where the cationic polymer is a copolymer based on dimethylaminoethyl methacrylate, butyl methacrylate, and methyl methacrylate.

In certain embodiments, the present disclosure provides a method of preparing a dosage form that further comprises combining the particulate populations in a tablet, a tablet-in-tablet, a bilayer tablet, or a capsule dosage form.

The present disclosure also provides a method for providing overdose protection from an opioid overdose, the method comprising orally administering to a subject a solid extended release multi-particulate dosage form with abuse deterrent and overdose protection characteristics comprising: (a) a first population of particulates comprising a therapeutically effective amount of at least one opioid embedded in a polymer matrix, a primary functional coat layer (FC 1) over the polymer matrix, a secondary functional coat layer (FC 2) over FC 1, and an over coat over FC 2, wherein FC 1 comprises a nonionic water-insoluble polymer and, optionally, at least one of a cationic polymer, a nonionic water-soluble polymer, and a water-soluble plasticizer; wherein FC 2 comprises at least one of a cationic polymer, a nonionic water-soluble polymer, and a water-soluble plasticizer and, optionally, a nonionic water-insoluble polymer; and wherein the over coat comprises a nonionic water-soluble polymer; and (b) a second population of particulates comprising an alkaline agent and a pH-stabilizing agent, wherein the dosage form provides an extended release of the opioid for a period of at least about 4 hours, and releases less than about 40% by weight of the opioid or a pharmaceutically acceptable salt thereof from the dosage form at about 1 hour; and wherein, when two or more dosage units are consumed, the alkaline agent raises the gastric pH and the pH-stabilizing agent maintains the elevated pH to further extend the release of the opioid from the dosage form.

The present disclosure also provides a method for providing analgesia by administering an extended release opioid dosage form in an overdose protection formulation without impeding release of the opioid when taken as directed, the method comprising orally administering to a subject a solid extended release multi-particulate dosage form with abuse deterrent and overdose protection characteristics comprising: (a) a first population of particulates comprising a therapeutically effective amount of at least one opioid embedded in a polymer matrix, a primary functional coat layer (FC 1) over the polymer matrix, a secondary functional coat layer (FC 2) over FC 1, and an over coat over FC 2, wherein FC 1 comprises a nonionic water-insoluble polymer and, optionally, at least one of a cationic polymer, a nonionic water-soluble polymer, and a water-soluble plasticizer; wherein FC 2 comprises at least one of a cationic polymer, a nonionic water-soluble polymer, and a water-soluble plasticizer and, optionally, a nonionic water-insoluble polymer; and wherein the over coat comprises a nonionic water-soluble polymer; and (b) a second population of particulates comprising an alkaline agent and a pH-stabilizing agent, wherein the dosage form provides an extended release of the opioid for a period of at least about 4 hours, and releases less than about 40% by weight of the opioid or a pharmaceutically acceptable salt thereof from the dosage form at about 1 hour; and wherein, when two or more dosage units are consumed, the alkaline agent raises the gastric pH and the pH-stabilizing agent maintains the elevated pH to further extend the release of the opioid from the dosage form.

### 4. BRIEF DESCRIPTION OF THE FIGURES

FIG. 1 depicts a schematic representation of Active Granules according to certain embodiments of the present disclosure.

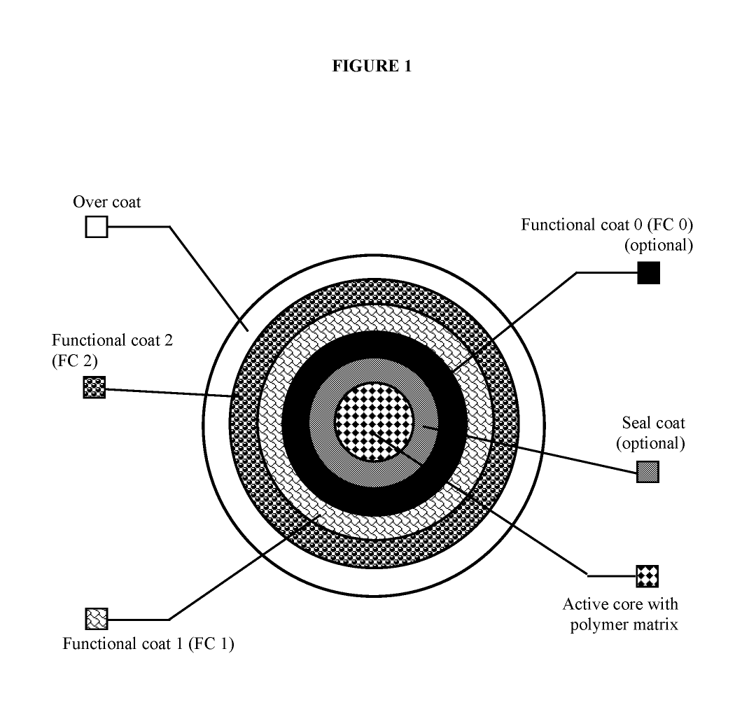

FIG. 2 shows a dissolution profile of oxycodone hydrochloride extended release tablets, 40 mg, 1 tablet vs. 6 tablets, in a two-stage dissolution method: a first stage comprising dissolution medium at pH 1.6 for 30 minutes, followed by a second stage comprising dissolution medium at pH 6.8 for an additional 330 minutes.

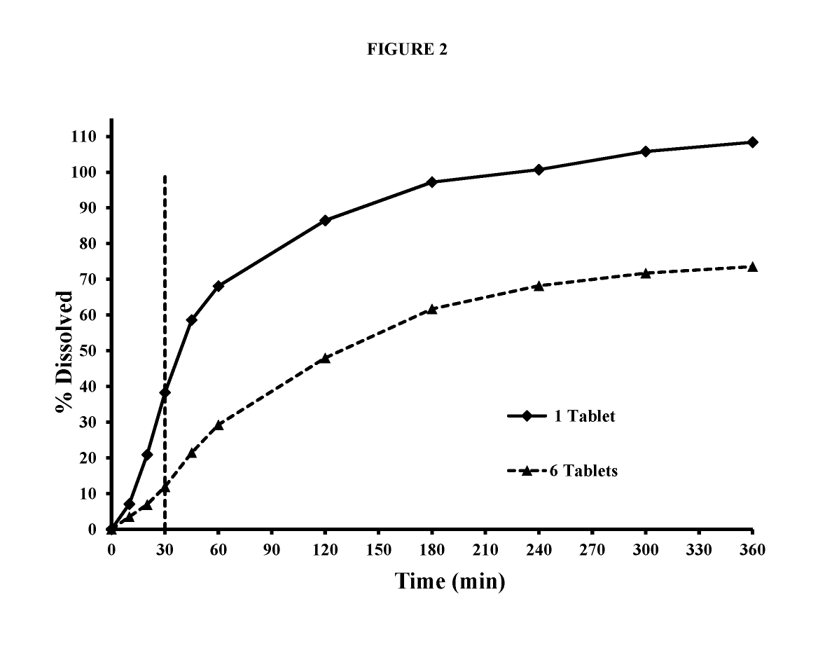

FIG. 3 shows a dissolution profile of hydrocodone bitartrate extended release tablets, 20 mg, 1 tablet vs. 6 tablets, in a two-stage dissolution method: a first stage comprising dissolution medium at pH 1.6 for 30 minutes, followed by a second stage comprising dissolution medium at pH 6.8 for an additional 330 minutes.

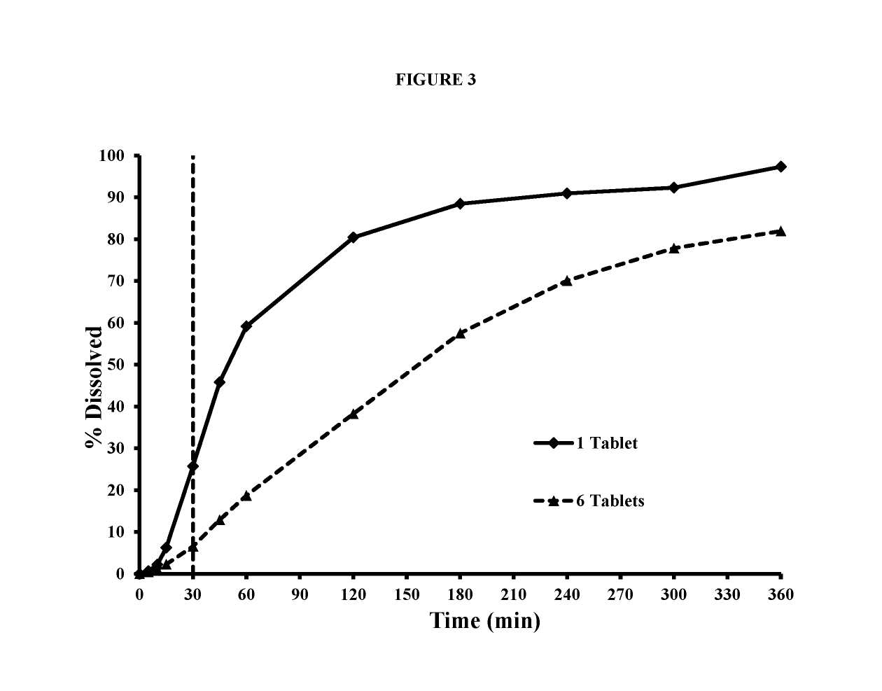

### 5. DETAILED DESCRIPTION

To date, there remains a need for improved ER abuse-deterrent formulations of abuse-prone drugs that make it difficult, if not impossible, for individuals to experience the dangerous consequences (including respiratory depression and death) of taking the dosage forms in a manner other than that intended by the manufacturer. Further, there remains a need for ER abuse-deterrent formulations of abuse-prone drugs that can maintain an ER profile even after being subjected to methods of abuse, such as heating and/or grinding. In addition, as provided herein, there remains a need for formulations of opioids that can provide an ER profile while simultaneously providing overdose protection when consumed in a manner other than intended by the manufacturer (e.g., multi-tablet dosing in excess of the prescribed number). The present disclosure provides improved solid oral ER multi-particulate pharmaceutical dosage forms containing at least two different populations of particulates. In certain embodiments, the ER pharmaceutical multi-particulate dosage forms contain at least three different populations of particulates. In certain embodiments, the ER pharmaceutical multi-particulate dosage forms contain at least four, at least five, or at least six different populations of particulates. Each population of particulates is designed for a specific function to accomplish the desired combination of abuse deterrence and overdose protection.

In certain embodiments of the disclosure, the ER pharmaceutical dosage forms contain at least a population of Active Particulates (i.e., Active Granules or Active Pellets), which is a crush-resistant particulate population comprising at least: (1) a core with an active agent (e.g., the polymer matrix of Active Granules) or a core with a coating of active agent (e.g., Active Pellets); (2) a functional coat layer(s) that provides a controlled release of the active agent in an aqueous or nonaqueous environment, as well as provides overdose protection (ODP) features of Active Particulates in the event of overdose (e.g., two or more dosage units). The functional coat layers (FCs) can include: FC 0 (optional); FC 1, coated on top of FC 0 (when present); and FC 2, coated on top of FC 1; these various FC layers provide a combination of controlled release, extended release, and ODP features to the Active Particulates.

In certain embodiments, the Active Particulate further includes a seal coat between the core (containing, or coated with, an active agent) and any layer that comprises a cationic polymer. For example, a seal coat is required between the core and FC 1 when FC 1 includes at least a portion of cationic polymer; a seal coat also is required between the core and FC 0 (when present), as FC 0 comprises a cationic polymer. In certain embodiments, FC 2 provides additional ODP by preventing or further slowing the release of active agent in an aqueous or nonaqueous environment with a pH above about 5. In certain embodiments, the Active Particulates further include an over coat to prevent the degradation of a certain ingredient(s) present in FC 2 and maintain the controlled release of active agent. In certain embodiments, the over coat prevents/reduces the interaction of a certain ingredient(s) (e.g., a cationic polymer) present in FC 2 with the alkaline agent present in the Triggering Particulates.

In certain embodiments, the dosage form further comprises Triggering Particulates (e.g., Triggering Granules) containing an alkaline agent that increases the pH of the aqueous or nonaqueous solution to above 5 in the presence of, e.g., two or three or more dosage units. In certain embodiments, the Triggering Particulates also contain a pH-stabilizing agent that maintains the increased pH above 5 for up to thirty minutes, or 45 minutes, or one hour, or 1.5 hours, or two hours. In certain embodiments, the increase in pH above 5 prevents or further slows the release of the active agent from the Active Particulates.

In certain embodiments, the extended release pharmaceutical dosage form further comprises a population of Viscosity-Enhancing Particulates (e.g., Viscosity-Enhancing Granules), containing a viscosity-building polymer that increases the viscosity of the aqueous or nonaqueous solution if tampered with, or taken in doses above those prescribed, or in a manner inconsistent with the manufacturer's instructions.

In certain embodiments, the pharmaceutical compounds for use in the present disclosure are those at risk for accidental (e.g., unintentional) or intentional overdose by the oral route (multi-tablet dosing) or other misuse by another route (e.g., intravenous, nasal, oral, rectal routes, etc.). In certain embodiments, this technology can be applied to opioids and other pharmaceutical compounds having solubility of greater than about 100 microgram/ml of physiological fluids including, for example, gastrointestinal (GI) fluids or simulated GI fluids (SGF)

### 5.1. Definitions

The terms used in this specification generally have their ordinary meanings in the art, within the context of the present disclosure and in the specific context in which each term is used. Certain terms are discussed below, or elsewhere in the specification, to provide additional guidance to the practitioner in describing the compositions and methods of the present disclosure and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing,” and “comprising” are interchangeable and one of skill in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within three or more than three standard deviations, per the practice in the art. Alternatively, “about” can mean a range of up to 15%, up to 10%, up to 5%, or up to 1% of a given value. Alternatively, particularly with respect to biological systems or processes, the term can mean within an order of magnitude, or within 5-fold, or within 2-fold, of a value.

The term “pharmaceutically acceptable” means a material that is not biologically or otherwise undesirable, i.e., the material can be incorporated into a pharmaceutical composition administered to a patient without causing any undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the FDA.

The term “active agent,” “drug,” “compound,” “active pharmaceutical ingredient,” or “API” refers to a pharmaceutically active substance which includes, without limitation, drugs susceptible to abuse and/or overdose. In certain embodiments, the active agent has a solubility of greater than about 100 microgram/ml of physiological fluids (e.g., GI fluid, SGF). In certain embodiments, the active agent is an opioid analgesic.

The term “opioid analgesic” includes single compounds and mixture of compounds selected from the group of opioids that can provide an analgesic effect. For example, opioid analgesics can include, without limitation, an opioid agonist, a mixed opioid agonist/antagonist, and a partial opioid agonist. In certain embodiments, the opioid can be a stereoisomer, ether, salt, hydrate, or solvate thereof. Opioid is also meant to encompass the use of all such possible forms as well as their racemic and resolved forms thereof, and all tautomers as well. The term “racemic” refers to a mixture of equal parts of enantiomers; such mixture is optically inactive.

As used herein, the phrase “therapeutically effective amount” means that amount that provides the specific pharmacological response for which the agent is administered to a subject in need of such treatment, for whatever reason. It is emphasized that a therapeutically effective amount will not always be effective in treating the target conditions/diseases, even though such amount is deemed to be a therapeutically effective amount by those of skill in the art. For illustration only, exemplary doses and therapeutically effective amounts are provided below with reference to adult human subjects. Those skilled in the art can adjust such amounts in accordance with standard practices as needed to treat a specific subject and/or condition/disease.

As used herein, the term “pharmaceutically acceptable salts” should be ascribed its customary meaning and includes, but is not limited to, inorganic acid salts such as hydrochloride, hydrobromide, sulfate, phosphate, and the like; organic acid salts such as formate, acetate, trifluoroacetate, maleate, tartrate, and the like; sulfonates such as methanesulfonate, benzenesulfonate, p-toluenesulfonate, and the like; amino acid salts such as arginate, asparaginate, glutamate, and the like; metal salts such as sodium salt, potassium salt, cesium salt, and the like; alkaline earth metals such as calcium salt, magnesium salt, and the like; and organic amine salts such as triethylamine salt, pyridine salt, picoline salt, ethanolamine salt, triethanolamine salt, discyclohexylamine salt, N,N′-dibenzylethylenediamine salt, and the like.

The term “patient” means a subject who has presented a clinical manifestation of a particular symptom or symptoms suggesting the need for treatment, who is treated preventatively or prophylactically for a condition, or who has been diagnosed with a condition to be treated.

The term “subject” is inclusive of the definition of the term “patient.” As used herein “a subject in need” of treatment by the methods described herein includes any subject suffering from or at risk of developing pain as described herein. The subject can be any mammal, including humans, horses, cats, and dogs. In particular embodiments, the subject is a human.

The term “immediate release” or “IR” refers to dosage forms that are formulated to allow the drug to dissolve in the gastrointestinal contents/fluids with no intention of delaying or prolonging the dissolution or absorption of the drug when taken as prescribed or in a manner consistent with manufacturer's instructions.

The term “extended release” or “ER,” or any term with a similar meaning as known in the art, refers to dosage forms that are formulated to allow the drug (active agent) to be available over a greater period of time after administration, thereby allowing a reduction in dosing frequency, as compared to a drug presented as a conventional dosage form (e.g., immediate release). Extended release may relate to the time of release, the extent of release, the rate of release, and/or release of an active ingredient from a formulation at such a rate that when a dose of the active ingredient is administered in an extended release formulation, concentrations (levels) of the active ingredient are maintained within a desired range, but below toxic levels, over a selected period of time. In certain embodiments, the dosage form is suitable for once daily or twice daily administration. In certain embodiments, the dosage form, after administration to a human patient or a population of patients, provides a time-to-peak plasma concentration (Tₘₐₓ) of the active agent from about 3 to about 14 hours. In certain embodiments, when administered in vivo, the extended release formulation allows for a timely onset of action in addition to a useful/efficacious plasma concentration of an active ingredient, e.g., less than 80%, less than 75%, less than 70%, less than 65%, less than 60%, less than 55%, less than 50%, less than 45%, less than 40%, less than 35%, less than 30%, less than 25%, less than 20%, less than 15%, or less than 10% of the active ingredient being dissolved and/or released in about 60 minutes, to be maintained for a longer period than in the case of administration of immediate release forms.

The term “stabilizing agent” means a compound or composition that serves to minimize or reduce deterioration of one or more properties of a pharmaceutical composition of the present embodiments, especially where the one or more properties can serve to create or enhance the abuse-deterrent properties of the pharmaceutical composition.

The term “oxidative stabilizing agent” means a stabilizing agent that serves to minimize or reduce the oxidative degradation and loss of viscosity that would otherwise occur when a heat-labile gelling agent, such as a PEO polymer, is subjected to heat. The oxidative stabilizing agent can be heat-resistant, meaning it does not decompose under hot melt extrusion, melt granulation, and/or curing conditions, and/or other heat-related conditions as described, e.g., in the present embodiments. The oxidative stabilizing agent can suppress oxidative degradation of oxidative degradable matrix materials such as PEO polymers and oxidation-sensitive drugs in pharmaceutical dosage forms.

The term “particulate” refers to a discrete, small, repetitive unit of particles, granules, or pellets that include at least one excipient, and optionally an active agent.

The term “multi-particulate” refers to at least two different populations of particulates.

The term “dosage form” refers to an oral particulate or multi-particulate solid drug delivery system that, in the present technology, includes at least one or two different populations of particulates.

The term “dosage unit” refers to an individual tablet (e.g., single tablet, tablet-in-tablet, bilayer tablet, multilayer tablet, etc.), capsule, pill, or other solid dosage form.

The term “coat” refers to a coating, layer, membrane, film, or the like, and can partially, substantially, or completely surround, cover, or envelop a substance, particulate, granule, drug, dosage unit, or the like. For example, a coat can cover portions of the surface to which it is applied, e.g., as a partial layer, partial coating, partial membrane, or a partial film; it can, for example, be in the form of spheres and/or half spheres that partially, substantially, or completely surround, cover, or envelop a surface.

The term “surrounding” if used alone, without any qualifier, can be understood to mean “at least partially surrounding.”

The term “acid labile coat” refers to a coat comprising a component(s) that will dissolve or degrade (partially or completely) in an acidic environment (e.g. in a solution with an acidic pH). The acidic pH can be, for example, below 7, below 6, below 5, below 4, below 3, below 2, or below 1. Typically, the pH at which an acid labile coat of the present disclosure will dissolve is in the normal physiological pH of the stomach, such as from about 1 to about 5, from about 1 to about 4, from about 1 to about 3, or from about 2 to about 3. Typically, the acid labile coat dissolves or degrades more slowly, or to only a small extent, when present in a solution with a pH that is considered not acidic and/or less acidic (e.g., at a pH above 5, above 6, or above 7). It will be understood that the acid labile coat can be prepared and designed to dissolve or degrade (partially or substantially) within any desired pH range, and to not dissolve or degrade (partially or substantially) within any desired pH range. For example, the acid labile coat can be designed to dissolve at any pH, e.g., below about 5; above that level, dissolution is inhibited, reduced or slowed. As the pH increases, the dissolution/degradation can slow further, and can stop nearly completely.

The term “alkaline agent” can be used to refer to an excipient that acts to increase the pH of, e.g., the gastric fluid (e.g., roughly pH 1.2-4.5) to a pH greater than 5. For example, alkaline agent can refer to substances that are capable of increasing the pH to greater than 4.5, greater than 5, greater than 5.5, etc. It also refers to basic substances and substances that can convert an acidic environment to a less acidic or a basic environment. Typically, these agents, when present in a sufficient amount, are able to raise the pH of the stomach to beyond physiological levels and thereby prevent, reduce, or inhibit dissolution of an acid labile substance or coat. Examples of alkaline agents include: aluminum hydroxide, sodium hydroxide, potassium hydroxide, calcium hydroxide, magnesium hydroxide, aluminum oxide, sodium oxide, potassium oxide, calcium oxide, magnesium oxide, calcium carbonate, sodium carbonate, potassium bicarbonate, sodium bicarbonate, ammonia, tertiary sodium phosphate, diethanolamine, ethylenediamine, N-methylglucamine, L-lysine, and combinations thereof.

The term “pH-stabilizing agent” refers to salts of weak acids/weak bases that act to maintain or stabilize the elevated pH of gastric fluid caused by an alkaline agent (e.g., an alkaline agent of the present disclosure). For example, a pH-stabilizing agent maintains the pH of the gastric fluid at a pH greater than about 5 for a finite time.

The term “functional coating,” “functional coat,” “functional coat layer,” “FC,” or the like refers to a coating that affects the rate of release, in vitro or in vivo, of an active drug(s), e.g., an opioid(s). Such coatings or coats are sometimes referred to as “rate-limiting” or “rate-controlling”; the particular polymer(s) responsible for affecting the rate of release in the coating or coat can also be referred to as “rate-limiting” or “rate-controlling.”

The term “viscosity-building polymer” as used herein refers to a polymer or group of polymers that increase the viscosity of the aqueous or nonaqueous medium of dissolution if tampered with, or increase the viscosity of the GI fluid when taken in doses above those prescribed or in a manner inconsistent with the manufacturer's instructions.

The term “nonionic polymer” refers to a polymer that remains in a nonionic form (i.e., a polymer with no net electrical charge and/or in a pH-independent form) in an acidic or a basic medium.

The term “water-insoluble nonionic polymer” refers to a nonionic pH-independent polymer generally insoluble in water, physiological fluids, and ethanol.

The term “water-soluble nonionic polymer” refers to a nonionic pH-independent polymer generally soluble in water, physiological fluids, and ethanol.

The term “cationic polymer” refers to a cationic pH-dependent polymer, or a polymer that changes into a cationic form in an acidic medium; it contains one or more cationic groups in acidic medium, and is generally soluble in, e.g., gastric fluid or a simulated gastric fluid.

The term “mini-tablet” refers to a tablet with a diameter equal to or smaller than 3 mm. Such mini-tablets can be filled into a capsule or compressed into a large tablet, etc.

The term “abuse-deterrent formulation,” “abuse-deterrent composition,” “abuse-resistant formulation,” “abuse-resistant composition,” or “ADF” are used interchangeably to refer to an oral dosage form that reduces the potential for abuse (e.g., improper administration) but delivers a therapeutically effective dose when administered as directed. For example, these terms generally refer to a dosage form that can be at least resistant to crushing, grinding, breaking, milling, melting, separating, cutting, extracting, dose dumping (e.g., alcohol dose dumping), and/or solubilizing for injection purposes. Improper administration includes, without limitation, tampering with the dosage form and/or administering the drug by any route other than that instructed. For example, and without limitation, improper administration includes snorting, administration after heat treatment, oral administration after crushing, or parenteral administration after extraction with a solvent such as water, ethanol, isopropanol, acetone, acetic acid, vinegar, carbonated beverages, and the like, and combinations thereof.

The term “abuse” means the intentional, nontherapeutic use of a dosage form or active agent to achieve a desirable psychological or physiological effect. For example, these terms refer to tampering with the dosage form and/or administering the drug in a manner inconsistent with the manufacturer's instructions. Methods of tampering or abuse include, but are not limited to, crushing, grinding, breaking, milling, melting, separating, cutting, extracting, dose dumping (e.g., alcohol dose dumping), and solubilizing for injection purposes.

As used herein, “in a manner inconsistent with the manufacturer's instructions” is meant to include, but is not limited to, consuming amounts greater than amounts described on the label or prescribed by a licensed physician, and/or altering by any means (e.g., crushing, breaking, milling, melting, separating, etc.) the dosage forms such that the active agent can be crushed, ground, melted, cut, extracted, dose dumped (e.g., alcohol dose dumping), and/or solubilized for injection purposes.

The term “crush resistant” or “resistant to crushing” means, e.g., a granule or particulate (e.g., an Active Granule) that can deform but does not break into powder form when pressure>500 N is applied, when using, e.g., a suitable hardness tester.

The term “grinding” refers to a process of reducing one or more tablets into small fragments, e.g., in the form of powder using, for example, a pestle and mortar, or following a specific grinding pattern (e.g., two minutes grinding/one minute rest/two minutes grinding) using an electrical grinding means (e.g., a coffee grinder or IKA grinder).

The term “heat pretreatment” refers to subjecting a pharmaceutical composition to heating conditions, e.g., heating in an oven or in a microwave, before manipulation by mechanical and/or chemical means. The term “heat pretreatment” does not include curing.

The term “heat stability” refers to the stability of a pharmaceutical composition, as measured by resistance to a heat pretreatment-induced increase in, for example, syringeability, extractability, dissolution, and/or alcohol-dose dumping, upon mechanical and/or chemical manipulation, after subjecting the composition to heat pretreatment. Mechanical and/or chemical manipulations can include, but are not limited to, crushing, grinding, grating, cutting, milling, and/or alcohol-dose dumping. The term “enhanced heat stability” is a comparative term referring to a superior form of the heat stability of a pharmaceutical formulation, as defined above.

The term “resistance to drug segregation” refers to the property of a pharmaceutical composition resulting in decreased or negligible drug segregation. For example, a pharmaceutical composition exhibits resistance to drug segregation when the percentage of drug content in the fines fraction (and/or in the coarse fraction) of a ground tablet is close to that predicted from the composition of the tablet itself. For example, the percentage of drug content in the fines (or coarse) fraction in a pharmaceutical composition exhibiting a resistance to drug segregation can be in the range of about 80% to about 130% to that predicted from the composition of the tablet (e.g., about 100%). More specifically, the percentage of drug content in a fines (or coarse) fraction can be in the range of about 80% to about 130%, about 85% to about 125%, about 85% to about 120%, about 85% to about 115%, about 90% to about 110%, about 95% to about 105%, to that predicted from the composition of the tablet.

The term “resistant to alcohol extraction” is used to refer to dosage forms that, in certain embodiments, at least fulfill the condition that in vitro dissolution, when measured in, for example, a USP Apparatus 1 (basket) at 100 rpm in 900 ml simulated gastric fluid comprising 40% ethanol at 37° C., is provided which is characterized by the percent amount of active released at 30 minutes of dissolution that deviates no more than 40% from the corresponding in vitro dissolution measured in a USP Apparatus 1 (basket) at 100 rpm in 900 ml simulated gastric fluid at 37° C. without ethanol.

The term “overdose protection” or “ODP” refers to a dosage form that reduces the potential for the detrimental consequences of overdose but delivers a therapeutically effective dose when administered as directed or prescribed by a licensed physician.

The term “overdose” refers to the administration of the dosage form of the present disclosure in amounts or doses above those considered therapeutic (e.g., two or more dosage units; more than one dosage unit); in a manner inconsistent with manufacturer's instructions; or in a manner not prescribed. Overdose can be intentional or unintentional (e.g., accidental).

As used herein, use of phrases such as “decreased,” “reduced,” “diminished,” or “lowered” is meant to include at least a 10% change in the release of the active agent with greater percentage changes being preferred for reduction in abuse potential and overdose potential. For example, but without limitation, the change can be greater than 10%, 15%, 25%, 35%, 45%, 55%, 65%, 75%, 85%, 95%, 96%, 97%, 98%, 99%, or increments therein.

### 5.2. Active Particulates

Active Particulates of the disclosed subject matter contain one or more active agents. In certain embodiments, the Active Particulates are Active Granules, Active Pellets, or a combination thereof. In certain embodiments, the Active Particulates are Active Granules. In certain embodiments, the Active Granules can include an active agent, a polymer matrix that in certain embodiments can include a hydrophilic polyoxyethylene (PEO) polymer, cationic polymer and/or nonionic polymer, an antioxidant, a plasticizer, and/or a surfactant. In certain embodiments, the polymer matrix of, e.g., the Active Granules containing the active agent can be directly surrounded (optionally) by a seal coat. In certain embodiments, at least one functional coat layer surrounds the polymer matrix: a primary functional coat layer (FC 1) directly or indirectly surrounding the polymer matrix. In certain embodiments, at least two functional coat layers surround the polymer matrix: a primary functional coat layer (FC 1) directly or indirectly surrounding the polymer matrix, and a secondary functional coat layer (FC 2) coated on top of FC 1. In certain embodiments, additional coating layers can be present, for example, but not limited to, between the core and seal coat, seal coat and FC 1, etc. In certain embodiments, three functional coating layers are present: a functional coat layer (FC 0) directly over the polymer matrix (or directly over the seal coat, when present); a functional coat layer (FC 1) directly over FC 0; and a functional coat layer (FC 2) directly over FC 1. In certain embodiments, the coating layers can be present in alternative orders or layering schemes.

In certain embodiments, FC 0 comprises a cationic polymer and, optionally, a nonionic water-insoluble polymer. In certain embodiments, FC 1 comprises a nonionic water-insoluble polymer and, optionally, a pore former, e.g., a cationic polymer, a nonionic water-soluble polymer, and/or a water-soluble plasticizer or polymer. In certain embodiments, FC 2 comprises a cationic polymer and, optionally, a nonionic water-insoluble polymer. In certain embodiments, the seal coat is optional. In certain embodiments, Active Particulates further include an over coat with a nonionic water-soluble polymer, or a water-soluble plasticizer or polymer. FC 1 (when containing, e.g., EUDRAGIT® E PO as a pore former) and FC 2 (as well as FC 0, when present) provide overdose protection when coupled with the alkaline agent(s) and pH-stabilizing agent(s) contained in one of the other granules (e.g., Triggering Particulates) present in the abuse-deterrent ODP formulation tablets or capsules of the present disclosure.

### 5.2.1. Active Agents

In certain embodiments, the Active Particulates contain at least one active agent. In certain embodiments, different populations of Active Particulates contain different active agents.

As discussed in further detail herein, the Active Particulates can be coated with a series of functional coat layers (i.e., FC 0 (optional), FC 1, and FC 2). In certain embodiments, FC 0 comprises a cationic polymer that dissolves at a pH below 5 (e.g., at a pH of less than about 5). In certain embodiments, FC 1 comprises a nonionic water-insoluble polymer and, optionally, a cationic polymer, a nonionic water-soluble polymer (e.g., polyethylene glycol (PEG), hydroxypropyl methylcellulose (HPMC)), and/or a water-soluble plasticizer. In certain embodiments, FC 2 includes a cationic polymer that dissolves at a pH below 5 and, optionally, a nonionic water-insoluble polymer.

The pharmaceutically active agent is present in the dosage form in an amount effective for the intended therapeutic purpose. These amounts are well known in the art. Indeed, the doses at which any of the presently known active agents embraced by the present can be given safely and effectively for the intended therapeutic purpose are known to those of skill in the art. In certain embodiments, the active agent is present in an amount of from about 0.1% to about 95% w/w of the Active Particulate, excluding the weight of any coatings. In certain embodiments, the active agent is present in an amount of about 0.2% to about 90%, about 0.3% to about 85%, about 0.4% to about 80%, about 0.5% to about 75%, about 0.6% to about 70%, about 0.7% to about 65%, about 0.8% to about 60%, about 0.9% to about 55%, about 1% to about 50%, about 2.5% to about 45%, about 5% to about 40%, about 7.5% to about 35%, about 10% to about 30%, about 12.5% to about 25%, or about 15% to about 20% w/w of the polymer matrix embedded with active agent. In certain embodiments, the active agent is present in an amount of at least about 0.1%, at least about 0.2%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% w/w of the polymer matrix embedded with active agent.

In certain embodiments, the active agents are drugs prone to abuse, misuse, and/or overdose. In certain embodiments, the active agents can include, without limitation, members of the therapeutic categories such as analgesics, anti-inflammatory agents, anthelmintics, anti-arrhythmic agents, anti-bacterial agents, anti-viral agents, anticoagulants, anti-depressants, anti-diabetic agents, anti-epileptic agents, anti-fungal agents, anti-gout agents, anti-hypertensive agents, anti-malarial agents, anti-migraine agents, anti-muscarinic agents, anti-neoplastic agents, erectile dysfunction improving agents, immunosuppressants, anti-protozoa agents, anti-thyroid agents, anti-anxiolytic agents, sedatives, hypnotics, neuroleptics, β-blockers, cardiac inotropic agents, corticosteroids, diuretics, anti-Parkinsonian agents, gastrointestinal agents, histamine receptor antagonists, keratolytics, lipid-regulating agents, anti-angina agents, cox-2 inhibitors, leukotriene inhibitors, macrolides, muscle relaxants, nutritional agents, opioid analgesics, protease inhibitors, sex hormones, stimulants, anti-osteoporosis agents, anti-obesity agents, cognition enhancers, anti-urinary incontinence agents, nutritional oils, anti-benign prostate hypertrophy agents, essential fatty acids, nonessential fatty acids, and any combinations of two or more thereof.

In certain embodiments, the active agent can be an opioid in a free base form or a pharmaceutically acceptable salt thereof. For example, but not limited to, the opioid can be alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydromorphodone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, nomiethadone, nalorphine, nalbuphene, normorphine, norpipanone, opium, oxycodone, oxymorphone, pantopon, papaveretum, paregoric, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, tapentadol, tilidine, tramadol, pharmaceutically acceptable salts thereof.

In certain embodiments, the opioid is oxycodone, or a pharmaceutically acceptable salt thereof. In certain embodiments, the opioid is oxycodone hydrochloride. In certain embodiments, the opioid is hydrocodone, or a pharmaceutically acceptable salt thereof. In certain embodiments, the opioid is hydrocodone bitartrate. In certain embodiments, the opioid is hydromorphone, or a pharmaceutically acceptable salt thereof. In certain embodiments, the opioid is hydromorphone hydrochloride. In certain embodiments, the opioid is oxymorphone. In certain embodiments, the opioid is codeine, or a pharmaceutically acceptable salt thereof.

In certain embodiments, the active agents can include, but are not limited to, benzodiazepines (e.g., bromazepam, chlordiazepoxide, clorazepate, diazepam, estazolam, flurazepam, halazepam, ketazolam, lorazepam, nitrazepam, oxazepam, prazepam, quazepam, temazepam, triazolam), barbiturates (e.g., amobarbital, aprobarbital, butabarbital, butalbital, methohexital, mephobarbital, metharbital, pentobarbital, phenobarbital, secobarbital), and stimulants, such as amphetamines (e.g., amphetamine, dextroamphetamine resin complex, dextroamphetamine, methamphetamine, methylphenidate), as well as dronabinol, glutethimide, methylprylon, ethchlorovynol, ethinamate, fenfluramine, meprobamate, pemoline, levomethadyl, benzphetamine, chlorphentermine, diethylpropion, phentermine, mebutamate, chlortermine, phenylacetone, dronabinol, nabilone, chloral hydrate, ethclorovynol, paraldehyde, midazolam, and dextropropoxyphene, or pharmaceutically acceptable salts thereof.

Examples of pharmaceutically acceptable salt include, but are not limited to, citrate, oxalate, acetate, maleate, malonate, fumarate, succinate, tosylate, mesylate, hydrochloride, hydrobromide, sulfate, phosphate, methanesulfonate, toluenesulfonate or mixtures and/or forms thereof. Additional pharmaceutically acceptable salts can be found in P. H. Stahl and C. G. Wermuth, editors, Handbook of Pharmaceutical Salts: Properties, Selection and Use, Weinheim/Zürich:Wiley-VCH/VHCA, 2002, the disclosure of which is herein incorporated by reference in its entirety.

### 5.2.2. Active Pellets

In certain embodiments, the Active Particulates are Active Pellets. In certain embodiments, the Active Pellets include an active agent and functional coat layers (e.g., FC 1 and FC 2, and optionally, FC 0). In certain embodiments, the Active Pellets can further include a seal coat. In certain embodiments, the Active Pellets are coated with at least one functional coat layer FC 1. In certain embodiments, the Active Pellets are coated with two functional coat layers FC 1 and FC 2. In certain embodiments, FC 0 comprises a cationic polymer and, optionally, a nonionic water-insoluble polymer. In certain embodiments, FC 1 comprises a nonionic water-insoluble polymer and, optionally, a cationic polymer, a nonionic water-soluble polymer (e.g., PEG, HPMC), and/or a water-soluble plasticizer. In certain embodiments, FC 2 includes a cationic polymer that dissolves at a pH below 5 and, optionally, a nonionic water-insoluble polymer. In certain embodiments, the Active Pellet includes an over coat comprising a nonionic water-soluble polymer. In certain embodiments, FC 1 includes a nonionic water-insoluble polymer and a cationic polymer, the latter soluble in gastric fluids (e.g., pH 1.2-4.5). This configuration provides an extended release of active agent over a period of about 3 to about 14 hours.

In certain embodiments, the core of the Active Pellets can be preformed pellets. By way of example, but not limitation, the pellet core can be made from microcrystalline cellulose (MCC) and/or alkaline agents/ion exchange resins. In certain embodiments, the pellet core comprises MCC cellets containing cured PEO polymers.

In certain embodiments, the shape of the pellets can be round, oval, or oblong.

In certain embodiments, that pellet core has a density of from about 0.3 to about 1.0 mg/cm³.

In certain embodiments, the pellet core can be from about 25 mg to about 500 mg. In certain embodiments, the pellet core can be about 50 mg to about 475 mg, about 75 mg to about 450 mg, about 100 mg to about 425 mg, about 125 mg to about 400 mg, about 150 mg to about 375 mg, about 175 mg to about 350 mg, about 200 mg to about 325 mg, about 225 mg to about 300 mg, or about 250 mg to about 275 mg.

In certain embodiments, the pellet core can be from about 25% to about 90% w/w of the uncoated Active Pellet, i.e., the Active Pellet before being coated with the (optional) seal coat, the functional coats, and the over coat. In certain embodiments, the pellet core can be about 27.5% to about 87.5%, about 30% to about 85%, about 32.5% to about 82.5%, about 35% to about 80%, about 37.5% to about 77.5%, about 40% to about 75%, about 42.5% to about 72.5%, about 45% to about 70%, about 47.5% to about 67.5%, about 50% to about 65%, about 52.5% to about 62.5%, or about 55% to about 60% w/w of the uncoated Active Pellet.

In certain embodiments, the pharmaceutically active agent is present in the dosage form in an amount effective for the intended therapeutic purpose. These amounts are well known in the art. Indeed, the doses at which any of the presently known active agents embraced by the present can be given safely and effectively for the intended therapeutic purpose are known to those of skill in the art. In certain embodiments, the active agent is present in an amount of about 0.1% to about 95% w/w of Active Particulates (i.e., Active Pellets and/or Active Granules) before the addition of the (optional) seal coat, or any functional coat(s) (e.g., about 0.1% to about 95% w/w of the polymer matrix embedded with active agent). In certain embodiments, the active agent is present in amounts as described above for Active Particulates.

In certain embodiments, Active Pellets (e.g., opioid-containing Opioid Pellets) contain an active agent (e.g., an opioid) in an amount of about 0.1% to about 95% w/w of the uncoated Active Pellets, i.e., the Active Pellets before being coated with the (optional) seal coat and/or any functional coat(s). In certain embodiments, Opioid Pellets contain the opioid in an amount of about 0.2% to about 90%, about 0.3% to about 85%, about 0.4% to about 80%, about 0.5% to about 75%, about 0.6% to about 70%, about 0.7% to about 65%, about 0.8% to about 60%, about 0.9% to about 55%, about 1% to about 50%, about 2.5% to about 45%, about 5% to about 40%, about 7.5% to about 35%, about 10% to about 30%, about 12.5% to about 25%, or about 15% to about 20% w/w of the uncoated Opioid Pellet. In certain embodiments, the Opioid Pellets contain the opioid in an amount of at least about 0.1%, at least about 0.2%, at least about 0.3%, at least about 0.4%, at least about 0.5%, at least about 0.75%, at least about 1%, at least about 2.5%, at least about 5%, at least about 7.5%, at least about 10%, at least about 12.5%, at least about 15%, at least about 17.5%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% w/w of the uncoated Opioid Pellet.

In certain embodiments, the opioid is oxycodone, or a pharmaceutically acceptable salt thereof. In certain embodiments, the opioid is oxycodone hydrochloride. In certain embodiments, the opioid is hydrocodone, or a pharmaceutically acceptable salt thereof. In certain embodiments, the opioid is hydrocodone bitartrate. In certain embodiments, the opioid is hydromorphone, or a pharmaceutically acceptable salt thereof. In certain embodiments, the opioid is hydromorphone hydrochloride. In certain embodiments, the opioid is oxymorphone. In certain embodiments, the opioid is codeine, or a pharmaceutically acceptable salt thereof.

In certain embodiments, the active agent can be absorbed by the pellet core. In certain embodiments, the active agent can be coated or layered onto the pellet core. In certain embodiments, the active agent can be dissolved into a suitable solvent system to either be absorbed by the pellet core or sprayed onto the pellet core. In certain embodiments, the solvent is water, an alcohol, an organic liquid, or a combination thereof. In certain embodiments, the alcohol is a dehydrated alcohol. In certain embodiments, the solvent is a mixture of water and an alcohol. In certain embodiments, the solvent is a mixture of water and a dehydrated alcohol. In certain embodiments, the components of a solvent mixture can be added at the same time or in different steps or stages.

In certain embodiments, solvents that can be used in processes of preparing dosage forms of the present disclosure include, but are not limited to, water, methanol, ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulfoxide, N,N-dimethylformamide, tetrahydrofuran, and any mixtures thereof.

In certain embodiments, the active agent coating can also contain additives/excipients such as coloring agents, talc, and/or magnesium stearate, which are well known in the coating arts. In certain embodiments, the excipients added to the active agent solution can include, but are not limited to hydroxypropyl methylcellulose (HPMC) (e.g., METHOCEL™ E5 Premium LV), lactose, polyvinylpyrrolidone (PVP), magnesium stearate, and talc. In certain embodiments, the excipients can be present in an amount of about 0.1% to about 30% w/w of the uncoated Active Pellet. In certain embodiments, the Active Pellets contain excipients in an amount of about 0.2% to about 27.5%, about 0.3% to about 25%, about 0.4% to about 22.5%, about 0.5% to about 20%, about 0.6% to about 17.5%, about 0.7% to about 15%, about 0.8% to about 12.5%, about 0.9% to about 10%, about 1% to about 7.5%, or about 2.5% to about 5% w/w of the uncoated Active Pellet. In certain embodiments, the Active Pellets contain excipients in an amount of at least about 0.1%, at least about 0.2%, at least about 0.5%, at least about 1%, at least about 5%, at least about 10%, at least about 15%, at least about 20%, at least about 25%, or at least about 30% w/w of the uncoated Active Pellet. In certain embodiments, the Active Pellets can be made by coating the active agent upon the pellet core.

### 5.2.3. Active Granules

In certain embodiments, the Active Particulates are Active Granules. In certain embodiments, the Active Granules comprise a polymer matrix, which includes an active agent, at least one hydrophilic polymer including polyoxyethylene (PEO), an antioxidant, and a plasticizer, as well as (optionally) an anionic polymer or a cationic polymer, another nonionic water-soluble polymer, and/or a surfactant. In certain embodiments, the Active Granules include a seal coat layer (optional), FC 0 (optional), FC 1, and FC 2. In certain embodiments, the Active Particulates include an over coat, comprising a water-soluble nonionic polymer and surrounding the functional coat layers. In certain embodiments, at least one of FC 0, FC 1, and FC 2 includes a water-insoluble nonionic polymer and a cationic polymer. The latter behaves as a pore former at a pH below about 5, but swells and becomes permeable at a pH above about 5 (e.g., in intestinal fluids, or in gastric fluids with an elevated pH), thereby substantially preventing release of the active agent at higher pH. In certain embodiments, FC 1 includes a nonionic water-insoluble polymer and, optionally, a pore former, e.g., a cationic polymer, a nonionic water-soluble polymer, and/or a water-soluble plasticizer.

In certain embodiments, a therapeutically effective amount of the active agent and the polymer matrix are contained in an inner core. In certain embodiments, the Active Granules contain a plasticizer in the inner core, the outer coating layers (e.g., the seal coat, one or more of the functional coat layers, and/or the over coat), or both the inner core and the outer coating layers. In certain embodiments, the Active Granules contain a surfactant in the inner core.

In certain embodiments, the Active Granules contain the active agent in an amount of about 0.1% to about 95% w/w of the uncoated Active Granules. In certain embodiments, the Active Granules contain an opioid in an amount of about 0.1% to about 95% w/w of the uncoated Active Granules.

In certain embodiments, the opioid is oxycodone, or a pharmaceutically acceptable salt thereof. In certain embodiments, the opioid is oxycodone hydrochloride. In certain embodiments, the opioid is hydrocodone, or a pharmaceutically acceptable salt thereof. In certain embodiments, the opioid is hydrocodone bitartrate. In certain embodiments, the opioid is hydromorphone, or a pharmaceutically acceptable salt thereof. In certain embodiments, the opioid is hydromorphone hydrochloride. In certain embodiments, the opioid is oxymorphone. In certain embodiments, the opioid is codeine, or a pharmaceutically acceptable salt thereof.

In certain embodiments, the polymer matrix can comprise a nonionic polymer, a cationic polymer, and/or an anionic polymer. Representative cationic polymers include, but are not limited to, (meth)acrylic polymers and (meth)acrylic copolymers (e.g., copolymers of alkyl (meth)acrylates and copolymers of alkylamino(meth)acrylates); and quarternary ammonium (meth)acrylic polymers.

Representative nonionic polymers include, but are not limited to, a nonionic pH-independent copolymer of ethyl acrylate, methyl methacrylate and a low content of methacrylic acid ester with quaternary ammonium groups (ammonium methacrylate copolymer, Type A, NF) (e.g., EUDRAGIT® RL 100, RS100 (Evonik)); and nonionic pH-independent polymers such as hydroxypropyl cellulose (e.g., KLUCEL® E, L, J, G, M and H grades (Ashland)), hydroxypropyl methylcellulose (HPMC) (e.g., METHOCEL™ E, F, J, and K grades (Dow Chemicals)), hydroxyethyl cellulose (e.g., NATRASOL L, G, M, and H grades (Ashland)), ethyl cellulose (e.g., ETHOCEL™ 7FP, 10FP, 45FP, and 100FP (Dow Chemicals) and N7, N10, N14, N22, N50, and N100 grades (Ashland)), cellulose acetate butyrate (e.g., CAB-381-0.5 (Eastman)), and cellulose acetate (CA-398-3, CA-398-6, CA-398-100, and CA-398-30 (Eastman)); polyvinyl acetate polymers (e.g., polyvinyl acetate-polyvinylpyrrolidone (KOLLIDON® SR) and polyethylene oxide polymers (e.g., POLYOX® WSR coagulant, POLYOX® WSR-301, POLYOX® WSR-303). Exemplary PEO polymers include POLYOX® WSR N-80, POLYOX® WSR N-750, POLYOX® WSR N-3000, POLYOX® WSR-205, POLYOX® WSR N-1105, POLYOX® WSR N-12K, POLYOX® WSR N-60K, POLYOX® WSR N-301, POLYOX® WSR coagulant, POLYOX® WSR N-303. The exemplary PEO polymers provide different viscosities in an aqueous solution. In certain embodiments, the exemplary PEO polymer has an average molecular weight of about 1,000,000 (WSR-N-12K), about 4,000,000 (WSR-301), about 5,000,000 (WSR Coagulant), or about 7,000,000 (WSR-303).

Representative anionic polymers include, but are not limited to, carbomers (e.g., Carbopol 934P, Carbopol 971P, and Carbopol 974P), EUDRAGIT® L 100, EUDRAGIT® S 100, EUDRAGIT® L 100-55, cellulose acetate phthalate (CAP), polyvinyl acetate phthalate (PVAP), and hypromellose acetate succinate (HPMCAS).

Representative pH-dependent polymers include, but are not limited to, cationic pH-dependent release polymers that are soluble in gastric fluid, but hydrate and become permeable at a pH above 5.0. In certain embodiments, the cationic pH-dependent polymer matrix comprises EUDRAGIT® E PO which has a molecular weight about 47,000 and a glass transition temperature about 48° C.

Representative water-soluble plasticizers include, but are not limited to, triethyl citrate and PEG (e.g., MW from 400-8000).

In certain embodiments, the polymer matrix (i.e., the polymer matrix without the active agent embedded within) can be present in the Active Granules in a range from about 1.0% to about 95%, from about 15% to about 90%, or from about 30% to about 75% w/w based on the total weight of the uncoated Active Granule. In certain embodiments, the polymer matrix can be present in an amount of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% w/w based on the total weight of the uncoated Active Granule.

In certain embodiments, a plasticizer can be added to increase the elasticity of the polymer(s) in Active Granules, thereby making the granules crush resistant. In certain embodiments, the plasticizer, when present in the core of an Active Granule, produces a crush-resistant Active Granule. In certain embodiments, the plasticizer present in a functional coat layer(s) (i.e., FC 0 (when present), FC 1, and FC 2) produces crush-resistant functional coat layer(s) (i.e., the membrane coat remains intact or relatively intact after crushing or grinding).

In certain embodiments, the plasticizer is soluble in both aqueous and nonaqueous solvents that are commonly used to extract opioids and other abuse-prone drugs from commercial formulations. In certain embodiments, the plasticizer acts as an aversion agent. In certain embodiments, the plasticizer acts as a tissue irritant that causes discomfort if administered in conj unction with an active agent with which it is coextracted.

Representative plasticizers include, but are not limited to liquid esters, (e.g., triethyl citrate, propylene glycol, polyethylene glycols, triacetin, diethylene glycol monoethyl ether, dibutyl sebacate, and diethyl phthalate). In certain embodiments, the dielectric constant values of the plasticizer are in a range of about 5 to about 60. In other embodiments, the dielectric constant values of the plasticizer are in a range of about 10 to about 40.

In certain embodiments, the plasticizer can be present in an amount that is sufficient to make the Active Granules substantially crush-resistant, but not in an amount that negatively impacts the dissolution of the active agent when the dosage form is taken in a manner consistent with the manufacturer's instructions, or in a manner consistent with that prescribed. In certain embodiments, the plasticizer can be present in amounts that result in discomfort to the abuser when the plasticizer is co-eluted with the active agent and administered in a manner inconsistent with the manufacturer's and/or physician's instructions. In certain embodiments, the amount of plasticizer provides an adequate rubbery state and elongation property to the polymer to achieve crush-resistance, making it difficult to pulverize the Active Granules into a fine powder, thereby deterring abuse.

In certain embodiments, the plasticizer can be present in a range of about 0.1% to about 30% w/w of the uncoated Active Granules. In certain embodiments, the plasticizer can be present in a range from about 2.0% to about 15% w/w of the uncoated Active Granules. In certain embodiments, the plasticizer can be present in an amount of about 0.2% to about 27.5%, about 0.3% to about 25%, about 0.4% to about 22.5%, about 0.5% to about 20%, about 0.6% to about 17.5%, about 0.7% to about 15%, about 0.8% to about 12.5%, about 0.9% to about 10%, about 1% to about 7.5%, or about 2.5% to about 5% w/w of the uncoated Active Granule. In certain embodiments, the plasticizer can be present in an amount of at least about 0.1%, at least about 0.2%, at least about 0.5%, at least about 1%, at least about 5%, at least about 10%, at least about 15%, at least about 20%, at least about 25%, or at least about 30% w/w of the uncoated Active Granule. In certain embodiments, the plasticizer can be present in an amount of about 2%, about 3%, about 4%, about 6%, or about 8% w/w of the uncoated Active Granule.

In certain embodiments, the matrix of an Active Granule further comprises at least one surfactant. In certain embodiments, the pharmaceutically acceptable surfactants that are useful in the practice of the present disclosure have solubility in oils, co-solvents, or aqueous media. In certain embodiments, the surfactant component helps in modulating the solubility of the active agent. In certain embodiments, the surfactant helps to reduce the abuse potential by a dual mechanism. First, the surfactant of the Active Granules elicits an irritant response when administered “as is” by nasal route. Second, the surfactant of the Active Granules elicits an irritant response by co-eluting with the drug when extracted with commonly used solvents, such as aqueous and organic solvents, for the injection route. Surfactants produce tissue irritation when applied to nasal mucosa and will cause local irritation at an injection site. Furthermore, docusate sodium is commonly used as a stool softener/laxative, so while providing some relief for opioid-induced constipation at the intended dose, it can cause undesirable gastrointestinal effects if large quantities are ingested. Similar undesirable gastrointestinal effects can be obtained by ingesting large quantities of other surfactants. In certain embodiments, the surfactant is present in an amount that results in discomfort to the abuser when the surfactant is co-eluted with the pharmaceutically active agent. The hydrophilic-lipophilic balance (“HLB”) values of the surfactants useful in the present disclosure are in a range of about 4 to about 30.

Types of surfactants that can be useful in the practice of the present disclosure include nonionic surfactants (e.g., esters of fatty acids, especially of C₈-C₂₄, and preferably of C₁₆-C₂₂, and fatty acid esters of polyols such as glycerol or sorbitol); sorbitan fatty acid esters ethoxylated with from 2 to 30 moles of ethylene oxide; polyethylene glycol fatty acid esters; polyethyleneglycol esters and polyethyleneglycol ethers; and polyethoxylated carboxylic acids (e.g., PEG-35 castor oil, PEG-40 castor oil, steareth-2 (e.g., Brij 72, Uniqema), steareth-21 (e.g., Brij 721, Uniqema), ceteareth-25 (e.g., Cremophor A25, BASF Cooperation), PEG-7 hydrogenated castor oil (e.g., Cremophor WO7, BASF Cooperation), and PEG-30 dipolyhydroxystearate (e.g., Arlacel P 135, Uniqema)); block copolymers based on ethylene oxide and propylene oxide (e.g., PLURONIC® (e.g., 188 or 407 (BASF)); dioctyl sodium sulfosuccinate (docusate sodium); sodium lauryl sulfate; PEG-32 glyceryl laurate; PEG-32 glyceryl palmitostearate; PEG-8 glyceryl caprylate/caprate; PEG-6 glyceryl caprylate/caprate; macrogol 15 hydroxystearate; polyoxyethylene 20 sorbitan monolaurate (polysorbate 20); polyoxyethylene 20 sorbitan monooleate (polysorbate 80); sorbitan monolaurate; sorbitan monooleate; and polyoxyl 40 stearate. Anionic surfactants (e.g., alkyl ether sulfates and sulfosuccinates) can also be useful. Alternatively, cationic and amphoteric surfactants such as phospholipids, lysophospholipids, and pegylated phospholipids can also be used. Additional useful surfactants include, vitamin E and derivatives thereof, (e.g., PEGylated derivatives of vitamin E such as tocopherol PEG succinate, tocopheryl polyethylene glycol sebacate, tocopheryl polyethylene glycol dodecanodioate, tocopheryl polyethylene glycol suberate, tocopheryl polyethylene glycol azelaate, tocopheryl polyethylene glycol citraconate, tocopheryl polyethylene glycol methylcitraconate, tocopheryl polyethylene glycol itaconate, tocopheryl polyethylene glycol maleate, tocopheryl polyethylene glycol glutarate, tocopheryl polyethylene glycol glutaconate, tocopheryl polyethylene glycol fumarate, tocopheryl polyethylene glycol phthalate, tocotrienol polyethylene glycol succinate, tocotrienol polyethylene glycol sebacate, tocotrienol polyethylene glycol dodecanodioate, tocotrienol polyethylene glycol suberate, tocotrienol polyethylene glycol azelaate, tocotrienol polyethylene glycol citraconate, tocotrienol polyethylene glycol methylcitraconate, tocotrienol polyethylene glycol itaconate, tocotrienol polyethylene glycol maleate, tocotrienol polyethylene glycol glutarate, tocotrienol polyethylene glycol glutaconate, tocotrienol polyethylene glycol fumarate and tocotrienol polyethylene glycol phthalate. See, e.g., U.S. Patent Publication No. 2014/0271593, the disclosure of which is hereby incorporated by reference in its entirety herein.

In certain embodiments, the surfactant can be present in a range of about 0.01% to about 15% w/w of the uncoated Active Granules. In certain embodiments, the surfactant can be present in a range from about 0.15% to about 5% w/w of the uncoated Active Granules. In certain embodiments, the surfactant can be present in an amount of about 0.025 to about 12.5%, about 0.05% to about 10%, about 0.075% to about 7.5%, about 0.1% to about 5%, about 0.25% to about 2.5%, or about 0.5% to about 1% w/w of the uncoated Active Granules. In certain embodiments, the surfactant can be present in an amount of about 0.2%, about 0.5%, about 2%, or about 2.2%, w/w of the uncoated Active Granules.

In certain embodiments, certain combinations of aversion agents (e.g., plasticizer and surfactant) can be used to deter abuse. Examples of such combinations include: triethyl citrate and docusate sodium; propylene glycol and docusate sodium; PEG-400 and docusate sodium; and PEG-400 and PEG-40 hydrogenated castor oil.

In certain embodiments, the Active Granules further contain an antioxidant. In certain embodiments, the antioxidants are present in an amount sufficient to suppress degradation of high molecular weight PEO upon hot melt extrusion (HME). Polymer degradation can result in an uncontrolled release profile, particularly when active material is embedded in a matrix of PEO; this can be another cause of oxidative degradation of pharmacologically active ingredients by, e.g., radicals. When adding an excipient, such as butylated hydroxytoluene (BHT), in order to attempt to stabilize high molecular weight PEO polymer, it should be taken into consideration that such an excipient should be stable at elevated temperatures, e.g., hot-melt extrusion temperatures used during manufacture of Active Granules, or used by an abuser to defeat the extended release properties of the dosage form. Antioxidants for use in the present disclosure include, but are not limited to, ascorbic acid and its salts, tocopherols, sulfite salts such as sodium metabisulfite or sodium sulfite, sodium sulfide, butylated hydroxyanisole, butylated hydroxytoluene, ascorbyl palmitate, and propyl gallate. In certain embodiments, the antioxidant can be present in a range of about 0.01% to about 2% w/w of the uncoated Active Granules. In certain embodiments, the antioxidant can be present in a range of about 0.025% to about 1%, about 0.05% to about 0.75%, about 0.075% to about 0.5%, or about 0.1 to about 0.75% w/w of the uncoated Active Granules. In certain embodiments, the antioxidant can be present in about 0.2%, about 0.3%, about 0.4%, or about 0.5% w/w of the uncoated Active Granules.

In certain embodiments, the Active Granules can be prepared in several ways known to those in the art, including hot-melt extrusion, film melt, granulation, melt granulation, extrusion spheronization, rotor or roller compaction. In certain embodiments, the Active Granules, containing PEO polymers, prepared by granulation, extrusion (e.g., HME), spheronization, rotor, or roller compaction process can require curing at a temperature above the melting point of the PEO polymers. In certain embodiments, the Active Granules, e.g., Opioid Granules, can be prepared by an HME process. In an HME process, a thermoplastic carrier polymer (e.g., nonionic polymer and/or cationic polymer) is combined with an active agent, a plasticizer, a surfactant, as well as any optional ingredients (e.g., an ion exchange polymer, alkaline buffering agent, and viscosity-building agent) to form a powdery mixture. The mixture is introduced into one or two rotating screws that convey the powder into a heated zone where shear forces compound the materials until a molten mass is achieved. Hot-melt extrusion equipment typically includes an extruder, auxiliary equipment for the extruder, downstream processing equipment, and other monitoring tools used for performance and product quality evaluation. The extruder is typically composed of a feeding hopper, barrels, single or twin screws, and the die and screw-driving unit. The auxiliary equipment for the extruder mainly includes a heating/cooling device for the barrels, a conveyer belt to cool down the product and a solvent delivery pump. The monitoring devices on the equipment include temperature gauges, a screw-speed controller, an extrusion torque monitor and pressure gauges. In certain embodiments, different shaped dies can be used. For example, extrudates can be produced by extruding the material through round-shaped dies into cooled rolls, wherein the extruded strands are cut into short cylinders using a pelletizer.

The pelletized extruded strands are subjected to an appropriate size reduction process(es) using co-mill or fitz mill or micropulverizer with coolant processing aids such as dry ice or liquid nitrogen.

In certain embodiments, the sizes of Active Granules, before or after attempted grinding, are significantly large enough to prevent the granules from being snorted. In certain embodiments, the mean size distribution of the Active Granules can be from about 125 m to about 1000 m, and in certain embodiments from about 250 m to about 750 μm (as measured by weight frequency distribution using sieving method). In certain embodiments, the mean particle size of the Active Granules is about 400 μm to about 600 μm. In certain embodiments, the mean particle size of the Active Granules is about 500 μm.

### 5.2.4. Seal Coat

In certain embodiments, the Active Particulates can be seal coated. The seal coat can be disposed between the polymer matrix core (i.e., the polymer matrix with active agent embedded within) and a functional coat layer (i.e., FC 1, or FC 0 (when present)). In certain embodiments, the seal coat can comprise a nonionic water-soluble polymer. In certain embodiments, the nonionic water-soluble polymer that is included in the seal coat is a cellulose ether polymer (e.g., a water-soluble methylcellulose polymer). In certain embodiments, the amount of the polymer ranges from about 5% to about 100%, from about 30% to about 95%, or from about 50% to about 75% w/w of the total weight of the composition of the seal coat (also noted within as “seal coat composition”). In certain embodiments, the amount of the polymer ranges from about 10% to about 95%, about 15% to about 90%, about 20% to about 85%, about 25% to about 80%, about 30% to about 75%, about 35% to about 70%, about 40% to about 65%, about 45% to about 60%, or about 50% to about 55% w/w of the total weight of the composition of the seal coat.

In certain embodiments, the composition of the seal coat can also include additional excipients, such as an anti-tacking agent (e.g., talc, magnesium trisilicate, colloidal silicon dioxide (e.g., CAB-O-SIL®)) and/or a plasticizer. In certain embodiments, the amount of additional excipients, when present, can range from about 0.1% to about 40%, or from about 0.5% to about 10% w/w of the total weight of the seal coat composition. In certain embodiments, the additional excipients are present at about 0.5% or about 4% w/w based on the total weight of the seal coat composition. In certain embodiments, the additional excipients are present at about 0.25% or about 35%, about 0.5% or about 30%, about 0.75% or about 25%, about 1% or about 20%, about 2.5% or about 15%, or about 5% or about 10% w/w based on the total weight of the seal coat composition.

In certain embodiments, the seal coat composition can also include an amount of the active agent, which can be therapeutically effective in and of itself, as well as a plasticizer and other excipients.

In certain embodiments, the seal coat can be present in a range of about 0.1% to about 40% w/w of the uncoated Active Particulates, i.e., the Active Particulates before being coated with the (optional) seal coat, the functional coat layers, and the over coat. In certain embodiments, the seal coat can be present in a range from about 5% to about 25% w/w of the uncoated Active Particulates. In certain embodiments, the seal coat can be present in an amount of about 5% or about 15% w/w of the uncoated Active Particulates. In certain embodiments, the seal coat can be present in a range of about 0.2% to about 37.5%, about 0.3% to about 35%, about 0.4% to about 32.5%, about 0.5% to about 30%, about 0.6% to about 27.5%, about 0.7% to about 25%, about 0.8% to about 22.5%, about 0.9% to about 20%, about 1% to about 17.5%, about 2.5% to about 15%, about 5% to about 12.5%, or about 7.5% to about 10% w/w of the total weight of the uncoated Active Particulates. In certain embodiments, the seal coat can be present in an amount of at least about 0.1%, at least about 0.2%, at least about 0.5%, at least about 1%, at least about 5%, at least about 10%, at least about 15%, at least about 20%, at least about 25%, at least about 30%, at least about 35%, or at least about 40% w/w of uncoated Active Particulates.

### 5.2.5. Functional Coat 0 (FC 0)

In certain embodiments, the Active Particulates are coated with a primary functional coat layer and a secondary functional coat layer (FC 1 and FC 2, respectively). In certain embodiments, the Active Particulates are coated with an additional functional coat layer preceding FC 1 and FC 2. This functional coat layer (i.e., FC 0) can be applied directly over the polymer matrix, or directly over the seal coat, when the latter is present. In certain embodiments, FC 0 comprises a cationic polymer that dissolves at a pH below 5 (e.g., at a pH of less than about 5).

In certain embodiments, FC 0 comprises a cationic polymer that is a dimethylaminoethyl methacrylate, butyl methacrylate, and methyl methacrylate copolymer. In certain embodiments, the dimethylaminoethyl methacrylate, butyl methacrylate, and methyl methacrylate copolymer is EUDRAGIT® E PO, EUDRAGIT® E 100, EUDRAGIT® E 12.5, or the like. In certain embodiments, FC 0 comprises a cationic polymer and a nonionic polymer. In certain embodiments, the nonionic polymer is a water-soluble polymer; in certain embodiments, the nonionic polymer is a water-insoluble polymer. In certain embodiments, the nonionic polymer is cellulose acetate.

### 5.2.6. Functional Coat 1 (FC 1)

In certain embodiments, FC 1 includes a nonionic water-insoluble polymer (e.g., a polymer that is not soluble in aqueous/physiological fluids and common organic solvents such as ethanol); optionally, FC 1 can also include a cationic polymer, a nonionic water-soluble polymer, and/or a water-soluble plasticizer, of which any or all can behave as pore formers.

In certain embodiments, FC 1 of the Active Particulates comprises at least a nonionic water-insoluble polymer, e.g., cellulose acetate, cellulose acetate-based polymers (e.g. OPADRY® CA, cellulose acetate butyrate, cellulose acetate propionate, and the like), polyvinyl acetate polymers, polyvinyl acetate-based copolymers (e.g., KOLLIDON® SR), ethylcellulose (e.g., ETHOCEL™), EUDRAGIT® RL 100, EUDRAGIT® RL PO, EUDRAGIT® RS 100, EUDRAGIT® RS PO, EUDRAGIT® NE 30 D, EUDRAGIT® NE 40 D, and the like, or a blend thereof; and at least one of a nonionic water-soluble polymer (e.g., PEG or HPMC), a cationic polymer (e.g., dimethylaminoethyl methacrylate, butyl methacrylate, and methyl methacrylate copolymer (e.g., EUDRAGIT® E PO)), or a water-soluble plasticizer (e.g., triethyl citrate and PEG (e.g., MW from 400-8000)).

In certain embodiments, FC 1 comprises at least cellulose acetate and a dimethylaminoethyl methacrylate, butyl methacrylate, and methyl methacrylate copolymer. In certain embodiments, the dimethylaminoethyl methacrylate, butyl methacrylate, and methyl methacrylate copolymer is EUDRAGIT® E PO.

EUDRAGIT® E PO is soluble in gastric fluid up to about pH 5. Above about pH 5, EUDRAGIT® E PO is hydrated and permeable. The uniqueness of the chemical properties of EUDRAGIT® E PO contributes to its dual roles in the overdose protection imparted by the present technology. In certain embodiments, the EUDRAGIT® E PO component functions as a pH-dependent pore former. It is soluble in aqueous fluids with a pH below about 5 (e.g., normal gastric fluid); thus, in certain embodiments, upon oral administration of an appropriate dose of the present inventive formulation, with the pH of the gastric fluid unmodified, EUDRAGIT® E PO, if present in FC 1, allows for the formation of pores in FC 1, and release of the opioid from the Active Particulates. In certain embodiments, when the pH of the gastric fluid is increased above about 5 (e.g., when two or more dosage units of the present disclosure are ingested), the EUDRAGIT® E PO present in FC 1 no longer dissolves, leading to decreased release (e.g., prevention of release) of the active opioid from the Active Particulates, thus accomplishing further control of opioid release. In certain embodiments, these processes function together to regulate (i.e., significantly reduce) the release of the active agent based on the pH of the gastric environment.

In certain embodiments, FC 1 includes a nonionic water-soluble polymer(s) (e.g., PEG, HPMC) as a pore former.

In certain embodiments, the ratio of water-insoluble cellulose polymers (e.g., cellulose acetate (“CA”), KOLLIDON® SR, and ETHOCEL™) to pore former (i.e., cellulose polymer:pore former) in FC 1 can be from about 80:20 to about 99.9:0.1 wt % ratio. In certain embodiments, the ratio of cellulose polymer to pore former can be about 85:15, about 90:10, about 93:7, about 95:5, about 95.5:4.5, about 96:4, about 96.5:3.5, about 97:3, about 97.5:2.5, about 98:2, about 98.5:1.5, about 99:1, or about 99.5:0.5 wt % ratio.

In certain embodiments, the nonionic water-insoluble polymer is a polyvinyl acetate (“PVA”) polymer or a PVA-based polymer or copolymer (e.g., KOLLIDON® SR) (collectively herein “PVA-based polymer”). In certain embodiments, the ratio of PVA-based polymer to pore former (i.e., PVA-based polymer:pore former) can be from about 80:20 to about 99.9:0.1 wt % ratio. In certain embodiments, the ratio of PVA-based polymer to pore former can be about 85:15, about 90:10, about 93:7, about 95:5, about 95.5:4.5, about 96:4, about 96.5:3.5, about 97:3, about 97.5:2.5, about 98:2, about 98.5:1.5, about 99:1, or about 99.5:0.5 wt % ratio.

In certain embodiments, if two or more dosage units are taken, release of the active agent from the dosage form is significantly reduced. In certain embodiments, the release is reduced by about 25%, 35%, 45%, 55%, 65%, 75%, 85%, 95%, 96%, 97%, 98%, 99%, or increments therein. In certain embodiments, the release is reduced from about 30% to about 90%, about 40% to about 80%, or about 50% to about 70%.

In certain embodiments, the composition of the functional coating can also include an anti-tacking agent (e.g., talc, magnesium trisilicate, colloidal silicon dioxide (e.g., CAB-O-SIL®)) and/or a plasticizer.

In certain embodiments, the functional coating prevents the extraction of the active agent in water and in water/alcohol mixtures.

In certain embodiments, FC 1 can be present in a range of about 5% to about 70% w/w of the uncoated or seal coated Active Particulates (i.e., the polymer matrix with active agent embedded within, also including the optional seal coat, if present). In certain embodiments, FC 1 can be present in a range of about 10% to about 65%, about 15% to about 60%, about 20% to about 55%, about 25% to about 50%, about 30% to about 45%, or about 35% to about 40% w/w of the uncoated or seal coated Active Particulates. In certain embodiments, FC 1 can be present in a range of about 5% to about 20%, about 6% to about 19%, about 7% to about 18%, about 8% to about 17%, about 9% to about 16%, about 10% to about 15.50%, or about 16.5% to about 17.5% w/w of the uncoated or seal coated Active Particulates. In certain embodiments, FC 1 can be present at about 20% w/w of the uncoated or seal coated Active Particulates.

In certain embodiments, the functional coating process can entail the following steps:
1. Add cellulose acetate and/or ETHOCEL™ to a solvent (e.g., acetone) in a suitable size stainless steel container and mix until clear solution is formed.
2. To the solution from step #1, add EUDRAGIT® E PO, PEG, and/or HPMC and purified water and mix for ˜5 minutes until a clear solution is formed (optional).
3. To the solution from step #2, add additional excipients (e.g., dibutyl sebacate and CAB-O-SIL®) and continue mixing until a homogenous dispersion is formed.
4. Coat the Active Particulate (e.g., using a Wurster fluid bed coater) to desired weight.
5. Dry the coated active particulates from step #4.

### 5.2.7. Functional Coat 2 (FC 2)

In certain embodiments, FC 1 is coated with FC 2 to further enhance the ODP features of the dosage form. In certain embodiments, FC 2 comprises a cationic polymer. In certain embodiments, FC 2 comprises a cationic polymer and a nonionic polymer. In certain embodiments, FC 2 comprises a cationic polymer (e.g., EUDRAGIT® E PO) and cellulose acetate. In certain embodiments, FC 2 comprises a ratio of cellulose acetate to EUDRAGIT® E PO of about 60:40, about 50:50, about 40:60, about 30:70, about 20:80, about 10:90, about 5:95, or about 0.5:99.5 wt % ratio, or any intermediate ratios therein.

In certain embodiments, the composition of FC 2 can also include an anti-tacking agent (e.g., talc, magnesium trisilicate, colloidal silicon dioxide (e.g., CAB-O-SIL®)) and/or a plasticizer.

In certain embodiments, FC 2 can be present in a range of about 5% to about 50% w/w of FC 1-coated Active Particulates (i.e., the polymer matrix with active agent embedded within, FC 1, and the optional seal coat, if present). In certain embodiments, FC 2 can be present in a range from about 10% to about 40% w/w of FC 1-coated Active Particulates. In certain embodiments, FC 2 can be present in a range from about 12.5% to about 37.5%, about 15% to about 35%, about 17.5% to about 32.5%, about 20% to about 30%, or about 22.5% to about 27.5% w/w of FC 1-coated Active Particulates.

### 5.2.8. Over Coat

In certain embodiments, the functional coated Active Particulates include an over coat to prevent/minimize the interaction of EUDRAGIT® E PO in, e.g., FC 2 with the alkaline agent present in the Triggering Particulates. The over coat can include a nonionic polymer (e.g., HPMC).

In certain embodiments, the composition of the over coat can also include additional excipients such as an anti-tacking agent (e.g., talc, magnesium trisilicate, colloidal silicon dioxide (e.g., CAB-O-SIL®)) and/or a plasticizer.

In certain embodiments, the over coat can be present in a range of about 5% to about 50% w/w of the functional coated Active Particulates (i.e., the polymer matrix with active agent (embedded within), the functional coats, and the optional seal coat, if present; or cellets coated with active agent, the functional coats, and the optional seal coat, if present). In certain embodiments, the over coat can be present in a range of about 10% to about 45%, about 10% to about 40%, about 10% to about 35%, about 10% to about 30%, about 15% to about 35%, about 15% to about 30%, about 15% to about 25%, about 15% to about 20%, about 20% to about 35%, about 20% to about 30%, about 25% to about 35%, or about 25% to about 30% w/w of the functional coated Active Particulates.

### 5.2.9. Crushability and Grindability Resistance

In certain embodiments, the Active Particulates (e.g., Active Granules) are at least partially crush-resistant and grind-resistant; in certain embodiments, they are substantially noncrushable and nongrindable, thereby making the active agent difficult to abuse using means and tools employed by drug abusers. For example, the Active Granules resist abuse via, but not limited to, crushing or grinding and swallowing; crushing or grinding and inhaling or insufflating nasally (“snorting”); crushing or grinding and smoking; and crushing or grinding, dissolving, and injecting (subcutaneously (i.e., skin popping), intravenously, or intramuscularly). In certain embodiments, the Active Granules cannot be ground or crushed into particles small enough to be effectively snorted or injected. In certain embodiments, the Active Granules cannot be pulverized into fine powder by mechanical grinding.

In certain embodiments, a plasticizer can be added to increase the elasticity of the polymer in Active Granules, thereby making the granules both crush-resistant and grind-resistant. In certain embodiments, a plasticizer (e.g., triethyl citrate) present in the core of an Active Granule makes the Active Granule crush-resistant and grind-resistant. In certain embodiments, the plasticizer present in the functional coat layers (FC 0 (when present), FC 1, and FC 2) makes the functional coat layers crush-resistant and grind-resistant (i.e., the coating layers remains intact after crushing or grinding).

In certain embodiments, the resistance of the Active Granules to crushing and grinding is provided by vitamin E, which prevents degradation of PEO during hot-melt extrusion (HME). Thus, heating during HME, in the presence of vitamin E, provides a curing process to the PEO in the core, making the plastic extrudates difficult to grind by conventional milling methods, as well as difficult to crush into powder. In addition, in certain embodiments, further resistance of the Active Granules to crushing and grinding is provided by the presence of PEO (with vitamin E) and HPMC in the core. In certain embodiments, Active Granules produced by HME and containing PEO and HPMC, followed by cryogenic milling, are not grindable by either common household grinders or analytical laboratory grinders, and are crush-resistant.

The crush-resistance of the Active Granules can be determined by a measurement of crushing strength required to deform the granules without any evidence of fragmentation or breaking into smaller pieces or powder using an Instron Tester or equivalent.

Abuse deterrence can be tested by examining the mean particle size following the physical manipulation of the Active Granule. For example, the Active Granules can be subjected to grinding in a coffee grinder, a mill, a mortar and pestle, a food processor, a blender, etc. For example, Active Granules can be placed in a coffee grinder (e.g., Hamilton Beach Coffee Grinder) and ground for several cycles (e.g., at a 10 cup setting for 8 cycles of 30 seconds each).

The mean particle size of the granules after grinding can be measured using sieve analysis that gathers granules of the same size into groups based on particle size. The weight of the particles in each group can be measured and compared to the unground sample.

In certain embodiments, the mean particle size after grinding the Active Granules is about 500 μm (with a range of about 250 μm to about 1000 m), as measured by weight frequency distribution using sieving method. In certain embodiments, the mean particle size after grinding the Active Granules is greater than about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, or about 700 μm.

### 5.3. Triggering Particulates

In certain embodiments, the Triggering Particulates can be Triggering Granules. In certain embodiments, the Triggering Granules can contain a combination of at least one alkaline agent (e.g., magnesium hydroxide, which increases the pH of, e.g., gastric fluid, from about 1.6 to greater than about 5) and/or at least one pH-stabilizing agent (e.g., di- and/or tricalcium phosphate, which maintains the newly increased pH of greater than about 5 for about one to about two hours). In certain embodiments, ingestion of one dosage unit (e.g., one tablet or capsule) results in little or no increase in pH of the gastric fluids. In certain embodiments, ingestion of multiple dosage units (e.g., two or more) results in the alkaline agent increasing the pH very rapidly above about 5. In certain embodiments, the pH-stabilizing agent acts to maintain or stabilize the increased pH caused by the alkaline agent. For example, ingestion of multiple dosage units results in (a) a rapid increase in pH caused by the alkaline agent; (b) modulation of pore formation in the functional coat; and (c) a decrease in the rate of release of the active agent (e.g., oxycodone) from the Active Particulate. In certain embodiments, upon ingestion of multiple dosage units (e.g., two or more), the pH of the gastric fluid increases very rapidly above a pH of about 5 in about 1 to about 8 minutes.

In certain embodiments, the alkaline agents for use in the Triggering Granules include, but are not limited to, aluminum hydroxide, sodium hydroxide, potassium hydroxide, calcium hydroxide, magnesium hydroxide, calcium carbonate, sodium carbonate, potassium bicarbonate, sodium bicarbonate, sodium oxide, potassium oxide, magnesium oxide, aluminum oxide, calcium oxide, ammonia, tertiary sodium phosphate, diethanolamine, ethylenediamine, N-methylglucamine, L-lysine, and combinations thereof. In certain embodiments, the alkaline agent is magnesium hydroxide.

In certain embodiments, the alkaline agent is present in an amount that when a single dosage unit is taken, it does not alter the pH of the gastric fluid. In certain embodiments, the alkaline agent is present in an amount from about 10% to about 90% w/w of total Triggering Granules. In certain embodiments, the alkaline agent is present in an amount from about 15% to about 85%, about 20% to about 80%, about 25% to about 75%, about 30% to about 70%, about 35% to about 65%, about 40% to about 60%, about 45% to about 55%, about 40% to about 70%, about 40% to about 80%, about 40% to about 90%, about 50% to about 70%, or about 70% to about 90% w/w of the total Triggering Granule. In certain embodiments, the alkaline agent is present in an amount of about 50% or about 60% w/w of total Triggering Granule.

In certain embodiments, the pH-stabilizing agent for use in the Triggering Granules include, but are not limited to, bismuth aluminate, bismuth carbonate, bismuth subcarbonate, bismuth subgallate, bismuth subnitrate, calcium phosphate, dibasic calcium phosphate, dihydroxyaluminum aminoacetate, dihydroxyaluminum glycine, magnesium glycinate, sodium potassium tartrate, tribasic sodium phosphate, tricalcium phosphate, and combinations thereof. In certain embodiments, the pH-stabilizing agent is a combination of dibasic calcium phosphate/tricalcium phosphate. In certain embodiments, the ratio of dibasic calcium phosphate to tricalcium phosphate (i.e., dibasic calcium phosphate:tricalcium phosphate) is about 1:1 to about 1:5 wt % ratio. In certain embodiments, the ratio of dibasic calcium phosphate to tricalcium phosphate is about 1:1.25 to about 1:4.75, about 1:1.5 to about 1:4.5, about 1:1.75 to about 1:4.25, about 1:2 to about 1:4, about 1:2.25 to about 1:3.75, about 1:2.5 to about 1:3.5, or about 1:2.75 to about 1:3.25 wt % ratio. In certain embodiments, the pH-stabilizing agent is anhydrous dibasic calcium phosphate.

In certain embodiments, the pH-stabilizing agent is present in an amount that when a single dosage unit is taken, it does not alter the pH of the gastric fluid, but when multiple dosage units are taken (e.g., two or more dosage units), the pH-stabilizing agent maintains the elevated pH levels caused by the alkaline agent. In certain embodiments, the pH-stabilizing agent is present in an amount sufficient to maintain or stabilize the pH of the gastric fluid above about 5 for up to about 5 hours. In certain embodiments, the pH-stabilizing agent is present in an amount sufficient to elevate and/or stabilize the pH of the gastric fluid above about 5 for up to about 5 hours. In certain embodiments, the pH-stabilizing agent is present in an amount sufficient to maintain the pH of the gastric fluid above about 5 for about 1 to about 2 hours. In certain embodiments, the pH-stabilizing agent is present in an amount sufficient to maintain the pH of the gastric fluid above about 5 for at least about 1 hour, at least about 1.25 hours, at least about 1.5 hours, at least about 1.75 hours, at least about 2 hours, at least about 2.25 hours, at least about 2.5 hours, at least about 2.75 hours, at least about 3 hours, at least about 3.25 hours, at least about 3.5 hours, at least about 3.75 hours, at least about 4 hours, at least about 4.25 hours, at least about 4.5 hours, at least about 4.75 hours, at least about 5 hours.

In certain embodiments, the pH-stabilizing agent is present in an amount from about 10% to about 60% w/w of total Triggering Granules. In certain embodiments, the pH-stabilizing agent is present in an amount from about 12.5% to about 57.5%, about 15% to about 55%, about 17.5% to about 52.5%, about 20% to about 50%, about 22.5% to about 47.5%, about 25% to about 45%, about 27.5% to about 42.5%, about 30% to about 40%, or about 32.5% to about 37.5% w/w of total Triggering Granules. In certain embodiments, the pH-stabilizing agent is present in an amount from about 15% to about 40% w/w of total Triggering Granules. In certain embodiments, the pH-stabilizing agent is present in an amount of about 20% or about 30% w/w of total Triggering Granules.

In certain embodiments, the alkaline agent and the pH-stabilizing agent (combined) (e.g., included in the Triggering Particulates) are present in an amount of less than 60% w/w (i.e., 60 wt %) of the total dosage form (or pharmaceutical composition). In certain embodiments, the alkaline agent and the pH-stabilizing agent (combined) are present in an amount of less than 60%, less than 55%, less than 50%, less than 45%, less than 44%, less than 43%, less than 42%, less than 41%, less than 40%, less than 39%, less than 38%, less than 37%, less than 36%, less than 35%, less than 34%, less than 33%, less than 32%, less than 31%, less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, or less than 15%, w/w of the total dosage form (or pharmaceutical composition).

In certain embodiments, the Triggering Granules include a binder, a disintegrant, filler (or diluents), and/or a lubricant.

Binders according to the present disclosure include, but are not limited to, hydroxypropyl celluloses in various grades, hydroxypropyl methylcelluloses in various grades, polyvinylpyrrolidones in various grades, copovidones, powdered acacia, gelatin, guar gum, carbomers, methylcelluloses, polymethacrylates, and starches.

Disintegrants according to the present disclosure include, but are not limited to, carmellose calcium, carboxymethylstarch sodium, croscarmellose sodium, crospovidone (crosslinked homopolymer of N-vinyl-2-pyrrolidone), low-substituted hydroxypropyl celluloses, sodium starch glycolate, colloidal silicon dioxide, alginic acid and alginates, acrylic acid derivatives, and various starches.

Lubricants according to the present disclosure include, but are not limited to, magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof.

The Triggering Granules can be prepared by any granulation method known to those of skill in the art. For example, the Triggering Granules can be made by dry granulation (e.g., direct blend, compacting and densifying the powders), wet granulation (e.g., addition of a granulation liquid onto a powder bed under the influence of an impeller or air), or melt granulation, roller compaction. The granulation product obtained can be milled to achieve uniform granules. The granules obtained can be subsequently coated with an aqueous dispersion.

In certain embodiments, the Triggering Granules manufacturing process can entail the following steps:
1. Add the alkaline agent (e.g., magnesium hydroxide) and pH-stabilizing agent(s) (e.g., anhydrous dibasic calcium phosphate/tri-calcium phosphate), and additional excipients to a high shear granulator and mix using an impeller and chopper at medium speed to achieve a uniform blend.
2. Subject the blend from step #1 to an appropriate granulation process such as hot-melt extrusion, melt granulation, roller compaction, high shear or low shear mixing.
3. Subject granules from step #2 to appropriate delumping or size reduction process (e.g., co-mill or fitz mill).
4. Dry the granules from step #3 using a fluid bed dryer or air oven until the LOD is <1%.
5. To the dried granules add extragranular excipients (e.g., anhydrous dibasic calcium phosphate/tri-calcium phosphate (˜50%), croscarmellose sodium, magnesium stearate, colloidal silicon dioxide) and mix using a V blender to achieve a uniform blend.
6. The final blend from step #5 can be compressed into tablet as such or can be blended with other granule types and then filled into a capsule or compressed into tablet.

In certain embodiments, the mean particle size distribution of the Triggering Granules is about 100 μm to about 1000 m. In certain embodiments, the mean particle size distribution of the Triggering Granules is about 150 μm to about 950 μm, about 200 μm to about 900 μm, about 250 μm to about 850 μm, about 300 μm to about 800 μm, about 350 μm to about 750 μm, about 400 μm to about 700 μm, about 450 μm to about 650 μm, or about 500 μm to about 600 μm. In certain embodiments, the mean particle size distribution of Triggering Granules is about 300 μm to about 800 μm.

### 5.4. Viscosity Enhancing Particulates

In certain embodiments, the Viscosity Enhancing Particulates can be Viscosity Enhancing Granules. Viscosity Enhancing Granules increase the viscosity of the dosage form when added to a solution, thus impeding the ability to extract the active agent from the dosage form or to pass the solution through a needle for injection purposes.

In certain embodiments, the increase in viscosity can also reduce the potential absorption of the active agent when taken in amounts in excess of two dosage units (e.g., two or more dosage units). As the viscosity of the solution in the GI tract increases, the active agent is eventually entrapped in a polymer gel matrix; thus, the dosage form is transformed from into an enhanced version of an extended release formulation (i.e., release of the opioid is further retarded by the overdose protection provided by the Viscosity Enhancing Granules). It is believed that the ingestion of increasing quantities of the formulation will not proportionally increase the maximum concentration (Cₘₐₓ) to reach the full potential of abusive effects (e.g., euphoria, sedation, and/or relaxation) of the active agent. In addition, it will take a longer time to reach maximum concentration (Tₘₐₓ). The results will be (1) a reduced desirability of deliberately abusing or overdosing on the active agent, and (2) a reduced likelihood of toxicity in the face of accidental overdose.

In certain embodiments, the Viscosity Enhancing Granules contain a viscosity-building polymer. In certain embodiments, the viscosity-building polymer is present in an amount that is sufficient to increases the viscosity of the surrounding fluid in the GI tract if multiple doses, e.g., two or more dosage units, are taken for abuse purpose and/or prevents syringeability by rapidly forming a gelatinous mass that resists passage through a needle when subjected to about 10 ml aqueous or nonaqueous media.

In certain embodiments, the Viscosity Enhancing Granules include a polymer matrix that can include a nonionic polymer (e.g., polyethylene oxide (PEO) polymers such as POLYOX® WSR coagulant, POLYOX® WSR-301, POLYOX® WSR-303) and/or pH-dependent polymers (e.g., anionic polymers such as carbomers (e.g., Carbopol 934P, Carbopol 971P, Carbopol 974P)).

In certain embodiments, Viscosity Enhancing Granules include an antioxidant, a plasticizer and/or a surfactant. In certain embodiments, the Viscosity Enhancing Granule matrix further includes a glidant (e.g., talc, colloidal silicon dioxide, magnesium trisilicate, powdered cellulose, starch, and tribasic calcium phosphate). In certain embodiments, the Viscosity Enhancing Granules matrix further includes a disintegrant.

In certain embodiments, the viscosity-building polymer is present in an amount that does not retard the release of the active agent from a single dose administration, but does slow down the release of the active agent after multiple dosage units are taken (e.g., two or more dosage units). In certain embodiments, the viscosity-building polymer is present in an amount from about 2% to about 60% w/w of total Viscosity Enhancing Granules. In certain embodiments, the viscosity-building polymer is present in an amount from about 5% to about 55%, about 10% to about 50%, about 15% to about 45%, about 20% to about 40%, or about 25% to about 35% w/w of total Viscosity Enhancing Granules. In certain embodiments, the viscosity-building polymer is present in an amount of about 15% or about 20% w/w of total Viscosity Enhancing Granules.

Viscosity Enhancing Granules can be prepared by any granulation method known to those of skill in the art. For example, the Viscosity Enhancing Granules can be made by dry granulation (e.g., direct blend, compacting and densifying the powders), wet granulation (e.g., addition of a granulation liquid onto a powder bed under the influence of an impeller or air), melt granulation, hot-melt extrusion, extrusion spheronization, or rotor granulation. The granulation product obtained can be milled to achieve uniform granules. The granules obtained can be subsequently coated with an aqueous dispersion.

In certain embodiments, the Viscosity Enhancing Granules manufacturing process can entail the following steps:
1. Add the polymer (e.g., POLYOX® WSR coagulant) and additional excipients (e.g. hydroxypropyl methylcellulose K200M, KOLLIDON® SR, docusate sodium, crospovidone/starch 1500) to a high shear granulator and mix to achieve a uniform powder mix using an impeller and chopper at medium speeds
2. Spray additional excipients in solution (e.g., (α-dl-Tocopherol solution and triethyl citrate) onto the powder mix from step #1 to achieve a uniform blend.
3. Subject the blend from step #2 to an appropriate granulation process such as hot-melt extrusion, rotor granulation, melt granulation, or extrusion spheronization.
4. Subject granules from step #3 to appropriate size reduction process using co-mill, fitz mill, micropulverizer with the aid of processing aids such as dry ice or liquid nitrogen known as cryomilling (cryogenic milling).
5. Spheronize the granules using rotor.
6. Granules can be optionally coated with enteric polymers such as EUDRAGIT® L100-55, hypromellose phthalate (HPMCP), hypromellose acetate succinate (HPMCAS), etc.

In certain embodiments, the mean particle size distribution of the Viscosity Enhancing Granules is about 125 μm to about 1000 m. In certain embodiments, the mean particle size distribution of the Viscosity Enhancing Granules is about 150 μm to about 950 μm, about 200 μm to about 900 μm, about 250 μm to about 850 μm, about 300 μm to about 800 μm, about 350 μm to about 750 μm, about 400 μm to about 700 μm, about 450 μm to about 650 μm, or about 500 μm to about 600 μm. In certain embodiments, the mean particle size distribution of Viscosity Enhancing Granules is about 250 μm to about 750 μm.

### 5.5. Particulate and Multi-Particulate Extended Release Dosage Forms

The present disclosure combines ADF and ODP properties in single solid oral extended release dosage form and thus addresses multiple health-related concerns, especially regarding habit-forming compounds for which there is a high propensity for abuse (e.g., opioids). In certain embodiments, abuse deterrence features and/or overdose protection features activate after the ingestion of two or more dosage units (e.g., two or more tablets/capsules). In certain embodiments, the abuse deterrence and/or overdose protection features activate when the multiple dosage units are taken together. In certain embodiments, the abuse deterrence and overdose protection activate when the multiple dosage units are taken in tandem. In certain embodiments, release of the active agent after ingesting one dosage unit results in the dosage form maintaining its extended release characteristics (i.e., there is no effect on the release of the active agent from the dosage form(s)). In certain embodiments, when two or more dosage units are taken together, release of the active agent from the dosage form is significantly reduced. In certain embodiments, the release is reduced by about 25%, 35%, 45%, 55%, 65%, 75%, 85%, 95%, 96%, 97%, 98%, 99%, or increments therein.

In certain embodiments, the ER pharmaceutical dosage form is a particulate (i.e., a single particulate) dosage form. In certain embodiments, the ER dosage form is a multi-particulate dosage form. In certain embodiments, the ER multi-particulate dosage form contains at least two different populations of particulates. In certain embodiments, the ER multi-particulate dosage form contains at least three different populations of particulates. In certain embodiments, the ER multi-particulate dosage form contains at least four, at least five, or at least six different populations of particulates. Each population of particulates is designed for a specific function to accomplish the desired combination of abuse deterrence and overdose protection qualities.

In certain embodiments, the pharmaceutical dosage forms contain at least one population of Active Particulates (e.g., Active Pellets and/or Active Granules) in combination with at least one population of Triggering Granules. In certain embodiments, the alkaline agent of the Triggering Granules increases the pH of the aqueous or nonaqueous solution to above about pH 5 in the presence of two or more dosage units, and the (optional) pH-stabilizing agent of the Triggering Granules maintains the increased pH above about 5 for up to about two hours. In certain embodiments, the functional coat layers (e.g., including partial and/or complete acid labile coat layers of the Active Particulates) only allows release of the active agent in an aqueous or nonaqueous environment with a pH below about 5, and prevents or slows the release of the active agent in a pH above about 5.

In certain embodiments, the pharmaceutical dosage forms contain at least one population of Viscosity Enhancing Granules. In certain embodiments, the pharmaceutical dosage forms contain at least one population of Active Particulates in combination with at least one population of Triggering Granules and at least one population of Viscosity Enhancing Granules. In certain embodiments, the Viscosity Enhancing Granules are present in an amount of from about 2% to about 50% of the total weight of the dosage form.

In certain embodiments, the pharmaceutical dosage forms contain at least one population of pH-dependent Viscosity Modifying Particulates. In certain embodiments, pH-dependent Viscosity Modifying Particulates are pH-dependent Viscosity Modifying Granules comprising a pH-dependent viscosity building polymer (e.g., carbomers, such as Carbopol 934P, Carbopol 971P, and Carbopol 974P). In certain embodiments, the pH-dependent viscosity building polymer can be present in an amount that does not retard the release of the active agent from a single dose administration, but does slow down the release of the active agent after multiple dosage units are taken. In certain embodiments, the pH-dependent Viscosity Modifying Granules can be present in an amount from about 0.5% w/w to about 15% w/w of the total weight of the dosage form. In certain embodiments, the pH-dependent Viscosity Modifying Granules can be present in an amount from about 0.75% w/w to about 12.5%, about 1% to about 10%, or about 2.5% to about 7.5% w/w of the total weight of the dosage form.

In certain embodiments, the pharmaceutical dosage forms contain at least one population of Active Particulates in combination with at least one population of Triggering Granules and at least one population of pH-dependent Viscosity Modifying Granules. In certain embodiments, the pharmaceutical dosage forms contain at least one population of Active Particulates in combination with at least one population of Triggering Granules, at least one population of Viscosity Enhancing Granules, and at least one population of pH-dependent Viscosity Modifying Granules.

In certain embodiments, the pharmaceutical dosage forms contain at least one population of Ion Exchange Resin Granules (e.g., Amberlite™ IRP 64, Amberlite™ IRP 69). The ion exchange resin of the Ion Exchange Resin Granules forms a matrix or complex with the drug and thus can alter the release of drug. In certain embodiments, the ion exchange resin can be present in an amount that binds to the active agent if the dosage form is tampered with, thereby preventing the release of the active agent from the dosage form. In certain embodiments, the Ion Exchange Resin Granules can be present in a concentration of about 1-5 M, and in certain embodiments about 1-3 M, based on the total molarity of the drug susceptible to abuse.

In certain embodiments, the pharmaceutical dosage forms contain at least one population of Active Particulates in combination with at least one population of Triggering Granules and at least one population of Ion Exchange Resin Granules. In certain embodiments, the pharmaceutical dosage forms contain at least one population of Active Particulates in combination with at least one population of Triggering Granules, at least one population of Viscosity Enhancing Granules, and at least one population of Ion Exchange Resin Granules. In certain embodiments, the pharmaceutical dosage forms contain at least one population of Active Particulates in combination with at least one population of Triggering Granules, at least one population of Viscosity Enhancing Granules, at least one population of pH-dependent Viscosity Modifying Granules, and at least one population of Ion Exchange Resin Granules.

In certain embodiments, the plurality of particulate populations can be blended with other excipients and additives and compressed into a tablet or loaded into a capsule. In certain embodiments, the tablet/capsule dosage form disintegrates rapidly once in contact with an aqueous medium. In certain embodiments, the capsule can be a soft or hard gelatin capsule. In certain embodiments, the capsule itself does not alter the release of the active agent.

In certain embodiments, the Active Particulates are present in an amount from about 10% to about 80% w/w of the total weight of the dosage form. In certain embodiments, the Active Particulates are present in an amount from about 15% to about 75%, about 20% to about 70%, about 25% to about 65%, about 30% to about 60%, about 35% to about 55%, about 40% to about 50%, about 50% to about 80%, about 60% to about 80%, about 70% to about 80%, about 10% to about 50%, about 20% to about 50%, about 30% to about 50%, or about 40% to about 50% w/w of the total weight of the dosage form. In certain embodiments, the Active Particulates are present in an amount of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% w/w of the total weight of the dosage form.

In certain embodiments, the Triggering Granules are present in an amount from about 20% to about 50% w/w of the total weight of the dosage form. In certain embodiments, the Triggering Granules are present in an amount from about 22% to about 48%, about 24% to about 46%, about 26% to about 44%, about 28% to about 42%, about 30% to about 40%, about 32% to about 38%, or about 34% to about 36% w/w of the total weight of the dosage form. In certain embodiments, the Triggering Granules are present in an amount from about 20% to about 50%, about 20% to about 48%, about 20% to about 46%, about 20% to about 44%, about 20% to about 42%, about 20% to about 40%, about 22% to about 50%, about 24% to about 50%, about 26% to about 50%, about 28% to about 50%, about 30% to about 50%, about 32% to about 50%, about 34% to about 50%, about 36% to about 50%, about 38% to about 50%, about 40% to about 50%, about 42% to about 50%, about 44% to about 50%, about 46% to about 50%, or about 48% to about 50% w/w of the total weight of the dosage form. In certain embodiments, the Triggering Granules are present in an amount of at least about 20%, at least about 22%, at least about 24%, at least about 26%, at least about 28%, at least about 30%, at least about 32%, at least about 34%, at least about 36%, at least about 38%, at least about 40%, or at least about 42%, at least about 44%, at least about 46%, at least about 48%, or at least about 50% w/w of the total weight of the dosage form.

In certain embodiments, the Viscosity Enhancing Granules are present in an amount from about 2% to about 50% w/w of the total weight of the dosage form. In certain embodiments, the Viscosity Enhancing Granules are present in an amount from about 5% to about 45%, about 10% to about 40%, about 15% to about 35%, or about 20% to about 30% w/w of the total weight of the dosage form.

In certain embodiments, the pH-dependent Viscosity Modifying Granules are present in an amount from about 0.5% to about 15% w/w of the total weight of the dosage form. In certain embodiments, the pH-dependent Viscosity Modifying Granules are present in an amount from about 0.75% to about 12.5%, about 1% to about 10%, or about 2.5% to about 7.5% w/w of the total weight of the dosage form.

In certain embodiments, the Ion Exchange Resin Granules are present in a concentration of about 1 M to about 5 M, and in certain embodiments about 1M to about 3 M, based on the total molarity of the drug susceptible to abuse.

In certain embodiments, a singular particulate population (e.g., a population of opioids) can be blended with other excipients and additives and compressed into a dosage form, e.g., a tablet, tablet-in-tablet, bilayer tablet, or multilayer tablet, or loaded into a capsule, or the like. In certain embodiments, additional solid ER dosage forms, including additional particulate, tablet, and/or capsule coating regimens, are contemplated. A nonlimiting set of examples follows.

In certain embodiments, the formulation is a single particulate dosage form comprising a single population of particulates containing at least one opioid, the particulates being compressed into a tablet or filled in a capsule, and at least one alkalinizing coat surrounding the tablet, or an alkaline agent in powder form surrounding the particulates in the capsule.

In certain embodiments, the multi-particulate dosage form is a two-particulate dosage form comprising a first population of particulates containing opioid, and a second population of particulates containing at least one alkaline agent and, optionally, at least one pH-stabilizing agent (Triggering Particulates); the two particulate populations being compressed into a tablet or filled in a capsule.

In certain embodiments, the tablet is further coated with an acid labile coat and, optionally, an alkalinizing coat on top of the acid labile coat.

In certain embodiments, Active Particulates contain an alkaline agent and, optionally, a pH-stabilizing agent in the polymer matrix.

In certain embodiments, the size of Active Particulates is increased (e.g., about 400 micrometers to about 2-3 mm) to provide enhanced control of release of active agent (e.g., opioid) in an ODP setting, while providing required and desired extended release.

In certain embodiments, the Active Particulates can have various functional coat layers or sets of functional coat layers (e.g., without limitation, combinations of FC 0, FC 1, and/or FC 2).

In certain embodiments, the Active Particulates have a seal coat (optional) on top of the polymer matrix.

In certain embodiments, the Active Particulates have an over coat on top of the functional coat layers.

In certain embodiments, capsules contain coated Active Particulates/mini-tablets (e.g., Opioid Particulates/mini-tablets coated with functional coat layers and an over coat on top of the functional coat layers), and Triggering Particulates/mini-tablets.

In certain embodiments, capsules contain Triggering Particulates/mini-tablets, and particulates/mini-tablets made from coated Active Particulates.

In certain embodiments, capsules contain Triggering Particulates/mini-tablets; and coated mini-tablets (e.g., mini-tablets/particulates coated with functional coat layers and an over coat on top of the functional coat layers) made from uncoated Active Particulates.

In certain embodiments, capsules contain particulates/mini-tablets of coated Active Particulates, and particulate/mini-tablets of Triggering Particulates.

In certain embodiments, capsules contain coated particulates/mini-tablets of uncoated Active Particulates, and particulates/mini-tablets of Triggering Particulates.

In certain embodiments, capsules contain coated Active Particulates/mini-tablets, and particulates/mini-tablets of Triggering Particulates.

In certain embodiments, capsules contain coated Active Particulates/mini-tablets (e.g., Active Pellets/mini-tablets (e.g., Opioid Pellets/mini-tablets coated with functional coat layers and an over coat on top of the functional coat layers)), and Triggering Particulates/mini-tablets.

In certain embodiments, capsules contain (1) mini-tablets/particulates comprising coated Active Particulates, and at least a portion of Triggering Particulates; and (2) a remaining portion of Triggering Particulates/mini-tablets.

In certain embodiments, capsules contain (1) coated mini-tablets/particulates comprising uncoated Active Particulates, and at least a portion of Triggering Particulates; and (2) a remaining portion of Triggering Particulates/mini-tablets.

In certain embodiments, the dosage form is a bilayer tablet comprising a first layer comprising coated Active Particulates, and a second layer comprising Triggering Particulates, and the two layers are compressed into a bilayer tablet.

In certain embodiments, the dosage form is a bilayer tablet comprising a first layer comprising a coated tablet comprising uncoated Active Particulates, and a second layer comprising Triggering Particulates, and the two layers are compressed into a bilayer tablet.

In certain embodiments, the dosage form is a tablet-in-tablet dosage form comprising an inner tablet comprising coated Active Particulates, and an outer tablet, partially or completely surrounding the inner tablet, comprising Triggering Particulates.

In certain embodiments, the dosage form is a tablet-in-tablet dosage form comprising an inner coated tablet comprising uncoated Active Particulates, and an outer tablet, partially or completely surrounding the inner tablet, comprising Triggering Particulates.

In certain embodiments, the dosage form is a capsule dosage form comprising coated or uncoated compressed tablets comprising an opioid, a nonopioid analgesic, and Triggering Particulates.

### 5.6. Syringeability and Extractability Resistance, and Heat Stability

In certain embodiments, the particulate and multi-particulate dosage forms of the present disclosure provide several additional abuse-deterrent properties, including syringeability resistance, extractability resistance, and heat stability. For example, the multi-particulate dosage forms resist abuse via, but not limited to, extraction of the active agent from the dosage form, syringeability of the active agent from the dosage form, and destabilization of the several abuse-deterrent attributes by various heat treatment-related manipulations. In certain embodiments, the combination of these additional properties, along with the aforementioned resistance to crushability and grindability of the Active Particulates, strongly deter or prevent abuse of the inventive multi-particulate dosage form.

In certain embodiments, the particulate and multi-particulate dosage forms of the present disclosure provide enhanced or superior heat stability to the pharmaceutical formulation. Without being bound by a theory, it is believed such superior heat stability is the result of several factors, including, but not limited to, the stabilization of PEO polymers in the presence of a suitable antioxidant that can withstand elevated temperatures. For example, such antioxidant provides enhanced heat stability to the PEO polymer during the hot melt extrusion or melt granulation process, and/or during the curing process, and/or the antioxidant prevents oxidative degradation of oxycodone, and/or the antioxidant prevents auto-oxidation of the PEO polymer.

In certain embodiments, resistance to extractability is provided by, e.g., carbomers in the Active Particulates of the dosage form. In certain embodiments, carbomers (such as Carbopol 934P, Carbopol 971P, Carbopol 974P), as well as other anionic polymers that are viscosity-enhancing agents, form a gel and increase viscosity in aqueous and/or alcoholic media, such as those media used by abusers attempting extraction of active agent from the dosage form. In certain embodiments, the gelling effect of, e.g., carbomers is greatly enhanced in the alkaline pH resulting from the release of alkaline agent (e.g., in attempted extraction, or in the stomach when two or more dosage units are consumed). In certain embodiments, carbomers in the core form gel and further diminish drug release, e.g., permeation from the core of Active Particulates into the GI fluid, or into aqueous media attempting to be drawn into a syringe. In certain embodiments, polymers present in the functional coat layers, e.g., EUDRAGIT® E PO, are also involved in decreasing permeation of the active agent from the Active Particulates, e.g., when extraction is attempted. The alkaline agent(s) present in the dosage form produces a rapid rise in the pH of aqueous media (e.g., in attempted extraction, or in the stomach when, e.g., two or more dosage units are consumed). The polymers present in the functional coat layers, e.g., EUDRAGIT® E PO, become insoluble in this alkaline media; thus the release of active agent from the dosage form is blocked.

In certain embodiments, resistance to syringeability is provided by polyoxyethylene (PEO) polymers and HPMC in the Active Particulates (e.g., in the core of the Active Granules). The gelling characteristics of these molecules, when exposed to aqueous media, provide resistance to syringeability, as the bore of the needle is blocked by the viscous nature of the diluted dosage form. In addition, carbomers included in the dosage form (e.g., in the core of the Active Granules) provide further resistance to syringeability; in response to the rapidly rising pH induced by, e.g., magnesium hydroxide in aqueous media, carbomer-based gelling is greatly enhanced, further diminishing drug release. Thus, less drug permeates into the aqueous media, and less drug is available to be drawn into the syringe. In certain embodiments, polymers present in the functional coat layers, e.g., EUDRAGIT® E PO, are also involved in resistance to syringeability. The alkaline agent(s) present in the dosage form produces a rapid rise in the pH of aqueous media. The polymers present in the functional coat layers, e.g., EUDRAGIT® E PO, become insoluble in this alkaline media and block release of active agent from the dosage form. Thus, attempts to draw fluid containing the active agent into a syringe are blocked as well.

In certain embodiments, resistance to syringeability and extractability are provided by one or more properties of the dosage form. For example, resistance is provided by the gelling characteristics of polyoxyethylene (PEO) polymers and HPMC in the Active Particulates (e.g., in the core of the Active Granules) when exposed to aqueous media; such gelling results in less drug permeating into the aqueous media, and less drug being available to be drawn into a syringe. In addition, carbomers included in the dosage form (e.g., in the core of the Active Granules) provide further resistance to syringeability; in response to the rapidly rising pH induced by MgOH₂ in aqueous media, carbomer-based gelling is greatly enhanced, diminishing drug release. Also in response to the elevated pH induced by MgOH₂, the functional coat layer(s) comprising a cationic polymer (e.g., EUDRAGIT® E PO) remain intact, further diminishing drug release from the dosage form. These unique properties of the dosage form are prominent in a physiological setting involving accidental overdose (or deliberate abuse) comprising ingestion of multiple dosage units (dosage forms).

In certain embodiments, the dosage form of the present technology exhibits enhanced or superior heat stability. In certain embodiments, the dosage form of the present technology maintains an extended release of the active agent (e.g., an opioid) after being subjected to heat treatment, including heat treatments commonly referred to as “crisping” by drug abusers. The heating can be carried out in a microwave or convection oven, or using a hot plate, lighter, candle, or the like.

In certain embodiments, the dosage form of the present technology maintains an extended release of the active agent (e.g., an opioid) after being subjected to heat treatment due to the stabilizing effects of an antioxidant (e.g., vitamin E) on PEO polymers during the HME process employed in preparation of the Active Granules. The enhanced stability of PEO polymers maintains the several abuse-deterrence attributes of the dosage form during heat treatments and heat manipulations, e.g., those intended to defeat those abuse-deterrence attributes.

For example, in certain embodiments, the dosage form (e.g., oral tablet) of the present technology maintains an extended release of the active agent after heating at about 100° C. for at least about 2 hours in an oven (e.g., a convection oven). In certain embodiments, the dosage form of the present technology maintains an extended release of the opioid after being heated in the center of a preheated microwave (e.g., 80° C.) for up to about 25 minutes (e.g., about 1 minute, about 3 minutes, about 5 minutes, about 8 minutes, about 11 minutes, about 14 minutes, about 16 minutes, about 18 minutes, about 21 minutes, about 23 minutes, or about 25 minutes, including intermediate lengths of time) at, e.g., 2000 watt power. In certain embodiments, the dosage form of the present technology maintains an extended release of oxycodone after being subjected to heating (e.g., crisping; heating at about 100° C. for at least about 2 hours in an oven; heating in a microwave at 1200 W for 3-14 minutes) comparable to, or with a deviation of no more than about 20% from, an extended release oxycodone oral tablet that is not subjected to such heating. In certain embodiments, the deviation can be no more than about 15%, about 10%, or about 5% from an extended release oxycodone dosage form that is not subjected to such heating.

### 5.7. Extended Release Profile

In certain embodiments, the present disclosure provides a solid oral extended release dosage form comprising an analgesically effective amount of an opioid or a pharmaceutically acceptable salt thereof and a sufficient amount of controlled release material to render the dosage form suitable for once-daily administration. The dosage form, after administration to a patient, provides a time-to-peak (Tₘₐₓ) plasma concentration of opioid in vivo from at least about 3 hours to at least about 14 hours, for example, at least about 4 hours, at least about 5 hours, at least about 6 hours, at least about 7 hours, at least about 8 hours, at least about 9 hours, at least about 10 hours, at least about 11 hours, at least about 12 hours, or at least about 13 hours.

In certain embodiments, the present disclosure provides a solid oral extended release dosage form comprising an analgesically effective amount of an opioid or a pharmaceutically acceptable salt thereof and a sufficient amount of controlled release material to render the dosage form suitable for twice-daily administration. The dosage form, after administration to a patient, provides a time-to-peak (Tₘₐₓ) plasma concentration of opioid in vivo from at least about 3 hours to at least about 14 hours, for example, at least about 4 hours, at least about 5 hours, at least about 6 hours, at least about 7 hours, at least about 8 hours, at least about 9 hours, at least about 10 hours, at least about 11 hours, at least about 12 hours, or at least about 13 hours.

In certain embodiments, the present disclosure provides a solid oral extended release dosage form comprising an analgesically effective amount of an opioid or a pharmaceutically acceptable salt thereof and a sufficient amount of controlled release material to render the dosage form suitable for once-daily administration. In certain embodiments, the dosage form provides an in vitro release of the opioid, when measured by the USP Paddle method at 50-75 rpm in 250 ml aqueous buffer, at a pH of 1.6 at 37° C. for 30 minutes, followed by in 306 ml (i.e., adding 6 ml of water and 50 ml of phosphate buffer) at a pH of 6.8 at 37° C. for an additional 330 minutes, of from about 10% to about 40% by weight of the opioid or a salt thereof released at about 1 hour. In certain embodiments, the dosage form provides an in vitro release of the opioid, by weight of the opioid or a salt thereof, of from about 40% to about 70% released at about 8 hours; from about 70% to about 90% released at about 16 hours; or greater than about 80% released at about 20 hours. In certain embodiments, the dosage form provides an in vitro release of the opioid, by weight of the opioid or a salt thereof, from about 10% to about 40% released at about 1 hour; greater than about 40% released at about 8 hours; greater than about 70% released at about 16 hours; or greater than about 80% released at 20 about hours.

In certain embodiments, the present disclosure provides a solid oral extended release dosage form comprising an analgesically effective amount of opioid or a pharmaceutically acceptable salt thereof and a sufficient amount of controlled release material to render the dosage form suitable for twice-daily administration. In certain embodiments, the dosage form provides an in vitro release of the opioid, when measured by the USP Paddle method at 50-75 rpm in 250 ml aqueous buffer, at a pH of 1.6 at 37° C. for 30 minutes, followed by in 306 ml (i.e., adding 6 ml of water and 50 ml of phosphate buffer) at a pH of 6.8 at 37° C. for an additional 330 minutes, by weight of the opioid or a salt thereof, of from about 10% to about 40% released at about 1 hour; from about 40% to about 70% released at about 4 hours; from about 70% to about 90% released at about 8 hours; or greater than about 80% released at about 10 hours. In certain embodiments, the dosage form provides an in vitro release of the opioid of from about 10% to about 40% released at about 1 hour; from about 40% to about 100% released at about 4 hours; or greater than about 80% released at about 8 hours.

In certain embodiments, the extended release dosage form of the present disclosure provides an in vitro release of the opioid, when measured by the USP Paddle method at 50-75 rpm in 250 ml aqueous buffer, at a pH of 1.6 at 37° C. for 30 minutes, followed by in 306 ml (i.e., adding 6 ml of water and 50 ml of phosphate buffer) at a pH of 6.8 at 37° C. for an additional 330 minutes, by weight of the opioid or a salt thereof, of less than about 50%; less than about 60%; less than about 70%; or less than about 75% released at about 1 hour.

In certain embodiments, the extended release dosage form provides an in vitro release of the opioid, when measured by the USP Paddle method at 50-75 rpm in 250 ml aqueous buffer, at a pH of 1.6 at 37° C. for 30 minutes, followed by in 306 ml (i.e., adding 6 ml of water and 50 ml of phosphate buffer) at a pH of 6.8 at 37° C. for an additional 330 minutes, by weight of the opioid or a salt thereof, of from about 10% to about 30%; from about 10% to about 35%; from about 10% to about 40%; from about 10% to about 45%; or from about 10% to about 50% released at about 1 hour.

In certain embodiments, the dosage form provides an in vitro release rate of hydrocodone, oxycodone, hydromorphone, or oxymorphone, or a pharmaceutically acceptable salt thereof, when measured by the USP Paddle method at 50-75 rpm in 250 ml aqueous buffer, at a pH of 1.6 at 37° C. for 30 minutes, followed by in 306 ml (i.e., adding 6 ml of water and 50 ml of phosphate buffer) at a pH of 6.8 at 37° C. for an additional 330 minutes, by weight of the opioid or a salt thereof, of from about 0% to about 40% released at about 1 hour; from about 10% to about 85% released at about 2 hours; or from about 20% to about 100% released at about 6 hours.

### 5.8. Methods

The disclosure provides methods related to the opioid pharmaceutical dosage forms and formulations.

In certain embodiments, the disclosure provides methods for preparing/manufacturing solid, oral, extended release, particulate and multi-particulate opioid dosage forms with abuse deterrent and overdose protection properties/characteristics. In certain embodiments, the method comprises preparing a first population of particulates comprising a therapeutically effective amount of at least one opioid embedded in a polymer matrix, a primary functional coat layer (FC 1) over the polymer matrix, a secondary functional coat layer (FC 2) over FC 1, and an over coat over FC 2, wherein FC 1 comprises a nonionic water-insoluble polymer and, optionally, at least one of a cationic polymer, a nonionic water-soluble polymer, and a water-soluble plasticizer, wherein FC 2 comprises at least one of a cationic polymer, a nonionic water-soluble polymer, and a water-soluble plasticizer and, optionally, a nonionic water-insoluble polymer, and wherein the over coat comprises a nonionic water-soluble polymer. In certain embodiments, the method further comprises preparing a second population of particulates comprising an alkaline agent and, optionally, a pH-stabilizing agent; and combining the first and second populations of particulates. In certain embodiments, the dosage form provides an extended release of the opioid for a period of at least about 4 hours. In certain embodiments, the dosage form releases less than about 40% by weight of the opioid or a pharmaceutically acceptable salt thereof from the dosage form at about 1 hour. In certain embodiments, when two or more dosage units are consumed, the alkaline agent raises the gastric pH and the pH-stabilizing agent (when present) maintains the elevated pH to further extend the release of the opioid from the dosage form.

In certain embodiments, the disclosure provides methods for providing overdose protection from an opioid overdose. In certain embodiments, the methods comprise orally administering to a subject in need of such treatment a solid, extended release, multi-particulate opioid dosage form with abuse deterrent and overdose protection characteristics as disclosed herein.

In certain embodiments, the disclosure provides methods for providing analgesia by administering an extended release opioid dosage form in an overdose protection formulation without impeding release of the opioid when taken as directed. In certain embodiments, the methods comprise orally administering to a subject in need of such treatment a solid, extended release, multi-particulate opioid dosage form with abuse deterrent and overdose protection characteristics as disclosed herein.

In certain embodiments, the disclosure provides methods of managing or treating pain with opioids, and discouraging their abuse or misuse. In certain embodiments, the methods comprise orally administering to a subject in need of such treatment a solid, extended release, multi-particulate opioid dosage form with abuse deterrent and overdose protection characteristics as disclosed herein.

In certain embodiments, the disclosure provides dosing regimens. In certain embodiments, the dosing regimens comprise orally administering to a subject in need of such treatment a solid, extended release, multi-particulate opioid dosage form with abuse deterrent and overdose protection characteristics as disclosed herein. In certain embodiments, the dosing regimen provides once-daily administration of the dosage form in appropriate dosages, as known to one of skill in the art, of the opioid, for example, oxycodone, hydrocodone, hydromorphone, oxymorphone, or pharmaceutically acceptable salts thereof. In certain embodiments, the dosing regimen provides twice-daily administration of the dosage form in appropriate dosages, as known to one of skill in the art, of the opioid, for example, oxycodone, hydrocodone, hydromorphone, oxymorphone, or pharmaceutically acceptable salts thereof.

The following examples are offered to more fully illustrate the present disclosure, but are not to be construed as limiting the scope thereof.

### 6. EXAMPLES

### Example 1: Crush-Resistant Active Granules

Active Granules are prepared for use in a 10 mg and 40 mg oxycodone hydrochloride dosage form.

TABLE 1
Formulation of Active Granules

| | Active Granule 1 | | Active Granule 2 | |
|---|---|---|---|---|
| Components | (% w/w) | mg/dose | (% w/w) | mg/dose |
| Oxycodone hydrochloride | 10.00 | 10.00 | 40.00 | 40.00 |
| POLYOX ® WSR coagulant | 79.54 | 79.54 | 52.27 | 52.27 |
| Triethyl citrate | 7.96 | 7.96 | 6.14 | 6.14 |
| Docusate sodium | 2.00 | 2.00 | 2.00 | 2.00 |
| α-dl-Tocopherol | 0.50 | 0.50 | 0.50 | 0.50 |
| Total | 100.0 | 100.0 | 100.0 | 100.0 |

### Manufacturing Procedure:

1. Oxycodone hydrochloride, POLYOX® WSR coagulant, and docusate sodium are added to a high-shear granulator and mixed into a uniform powder mix using an impeller and a chopper.
2. A solution of α-dl-tocopherol and triethyl citrate in isopropyl alcohol is sprayed onto the powder mix from step #1 to achieve a uniform blend.
3. The blend from step #2 is granulated by hot-melt extrusion.
4. The granules from step #3 are processed using cryomilling to a mean particle size of about 500 μm.
5. The granules from step #4 are spheronized using a rotor.

### Example 2: Crush-Resistant Active Granules

Active Granules are prepared for use in a 10 mg and 40 mg oxycodone hydrochloride dosage form.

TABLE 2
Formulation of Active Granules

| | Active Granule 3 | | Active Granule 4 | |
|---|---|---|---|---|
| Components | (% w/w) | mg/dose | (% w/w) | mg/dose |
| Oxycodone hydrochloride | 10.00 | 10.00 | 40.00 | 40.00 |
| POLYOX ® WSR coagulant | 63.64 | 63.64 | 33.64 | 33.64 |
| HPMC K200M | 12.89 | 12.89 | 9.41 | 9.41 |
| KOLLIDON ® SR | 6.44 | 6.44 | 4.71 | 4.71 |
| Triethyl citrate | 3.83 | 3.83 | 2.83 | 2.83 |
| Docusate sodium | 2.00 | 2.00 | 2.00 | 2.00 |
| α-dl-Tocopherol | 0.20 | 0.20 | 0.20 | 0.20 |
| CARBOPOL ® | 1.00 | 1.00 | 1.00 | 1.00 |
| Total | 100.0 | 100.0 | 100.0 | 100.0 |

### Manufacturing Procedure:

1. Oxycodone hydrochloride, POLYOX® WSR coagulant, HPMC K200M, KOLLIDON® SR, CARBOPOL®, and docusate sodium are added to a high shear granulator and mixed into a uniform powder mix using an impeller and a chopper.
2. A solution of α-dl-tocopherol and triethyl citrate in isopropyl alcohol is sprayed onto the powder mix from step #1 to achieve a uniform blend.
3. The blend from step #2 is granulated by hot-melt extrusion.
4. The granules from step #3 are processed using cryomilling to a mean particle size of about 500 μm.
5. The granules from step #4 are spheronized using a rotor.

### Example 3: Active Pellets

Active Pellets with microcrystalline cellulose (MCC) core (Cellets) are prepared for use in a 40 mg oxycodone hydrochloride dosage form.

TABLE 3
Formulation of Active Pellets

| | Active Pellets 1 | |
|---|---|---|
| Components | (% w/w) | mg/dose |
| Microcrystalline cellulose pellets (Cellets) | 82.64 | 300.00 |
| Oxycodone hydrochloride | 11.02 | 40.00 |
| METHOCEL ™ E5 Premium LV | 5.51 | 20.00 |
| Talc | 0.83 | 3.00 |
| Solvent system for coating: | | |
| Purified water | 30.00 | NA |
| Dehydrated alcohol | 70.00 | NA |
| Total | 100.0 | 363.00 |

### Manufacturing Procedure:

1. Oxycodone hydrochloride is added to dehydrated alcohol in a stainless steel container and mixed until it disperses uniformly.
2. After the oxycodone is uniformly dispersed, METHOCEL™ E5 is added gradually during continued mixing until it disperses uniformly.
3. Purified water is added to the dispersion from step #2 and mixed until a clear solution is formed.
4. Talc is added to the solution from step #3 and mixed for at least 30 minutes or until it is dispersed.
5. The microcrystalline cellulose pellets are coated using a Wurster fluid bed coater with an inlet air temperature of 40°-50° C. and sufficient air volume for fluidization. When the product temperature reaches 30° C., the dispersion from step #4 is sprayed onto the pellets while maintaining a temperature of 28°-30° C. and sufficient air volume for the fluidization until the target coating weight gain is reached.
6. The coated pellets from step #5 are dried.

### Example 4: Seal Coating of Granules

Active Granules are coated with a seal coat.

TABLE 4
Formulation of Seal Coated Granules

| | Seal Coated | |
|---|---|---|
| | Granules 1 | |
| Components | (% w/w) | mg/dose |
| Active Granules | 83.33 | 100.00 |
| (oxycodone hydrochloride | | |
| granules 1-4) | | |
| METHOCEL ™ E5 Premium LV | 14.82 | 17.78 |
| Dibutyl sebacate | 1.48 | 1.78 |
| CAB-O-SIL ® | 0.37 | 0.44 |
| Solvent system for coating: | | |
| Purified water | 30.00 | NA |
| Dehydrated alcohol | 70.00 | NA |
| Total | 100.00 | 120.00 |

### Coating Procedure:

1. METHOCEL™ E5 is added to dehydrated alcohol in a stainless steel container and mixed until it disperses uniformly.
2. To the dispersion from step #1, the purified water is added and mixed until a clear solution forms.
3. To the solution from step #2, dibutyl sebacate is added, followed by the addition of CAB-O-SIL® and continued mixing until a homogenous dispersion is formed.
4. The granules (any of Granules 1-4) are coated using a Wurster fluid bed coater with an inlet air temperature of 40°-50° C. and sufficient air volume for fluidization. When the product temperature reaches 30° C., the dispersion from step #3 is sprayed onto the granules while maintaining the product temperature of 28°-30° C. and sufficient air volume for the fluidization until the target coating weight gain is reached.
5. The coated granules from step #4 are dried.

### Example 5: Seal Coating of Pellets

Active Pellets with MCC core (Cellets) are coated with a seal coat.

TABLE 5
Formulation of Seal Coated Pellets

| | Seal Coated Pellets 1 | |
|---|---|---|
| Components | (% w/w) | mg/dose |
| Active Pellets (pellet 1) | 90.91 | 363.00 |
| METHOCEL ™ E5 Premium LV | 8.08 | 32.27 |
| Dibutyl sebacate | 0.81 | 3.23 |
| CAB-O-SIL ® | 0.20 | 0.80 |
| Solvent system for coating: | | |
| Purified water | 30.00 | NA |
| Dehydrated alcohol | 70.00 | NA |
| Total | 100.00 | 399.30 |

### Coating Procedure:

1. METHOCEL™ E5 is added to dehydrated alcohol in a stainless steel container and mixed until it disperses uniformly.
2. To the dispersion from step #1, purified water is added and mixed until a clear solution formed.
3. To the solution from step #2, dibutyl sebacate is added, followed by the addition of CAB-O-SIL® and continued mixing until a homogenous dispersion formed.
4. The pellets (Active Pellet 1) are coated using a Wurster fluid bed coater with an inlet air temperature of 40°-50° C. and sufficient air volume for fluidization. When the product temperature reaches 30° C., the dispersion from step #3 is sprayed onto the pellets while maintaining the product temperature of 28°-30° C. and sufficient air volume for fluidization until the target coating weight gain is reached.
5. The coated pellets from step #4 are dried.

### Example 6: Functional Coating (FC 1) of Granules

Seal coated Active Granules are coated with a functional coat (FC 1) comprising, e.g., KOLLIDON® SR, cellulose acetate, or ETHOCEL™, either alone or at a ratio of [KOLLIDON® SR, cellulose acetate, or ETHOCEL™] to [EUDRAGIT® E PO, PEG, or HPMC] of 95:5.

TABLE 6
Formulation of Functional Coated (FC 1) Granules

| | Functional Coated | | Functional Coated | |
|---|---|---|---|---|
| | Active Granules 1 | | Active Granules 2 | |
| Components | (% w/w) | mg/dose | (% w/w) | mg/dose |
| Seal coated granules | 83.33 | 120.00 | 83.33 | 120.00 |
| (granules from | | | | |
| Example 4) | | | | |
| Cellulose acetate or | 13.89 | 20.00 | 13.19* | 19.00 |
| ETHOCEL ™ | | | | |
| EUDRAGIT ® E PO, | NA | NA | 0.69* | 1.00 |
| PEG, or HPMC | | | | |
| CAB-O-SIL ® | 0.69 | 1.00 | 0.69 | 1.00 |
| Dibutyl sebacate | 2.08 | 3.00 | 2.08 | 3.00 |
| Solvent system for coating: | | | | |
| Acetone | 90.00 | NA | 90.00 | NA |
| Purified water | 10.00 | NA | 10.00 | NA |
| Total | 100.00 | 144.00 | 100.00 | 144.00 |

*Ratio of cellulose acetate or ETHOCEL ™:EUDRAGIT ® E PO, PEG, or HPMC (95:5)

### Coating Procedure:

1. Cellulose acetate or ETHOCEL™ is added to acetone in a stainless steel container and mixed until a clear solution is formed.
2. To the solution from step #1, purified water is added, [optionally with EUDRAGIT® E PO, PEG, or HPMC (see, e.g., Functional Coated Active Granules 2, above)], and mixed for ˜5 minutes until a clear solution formed.
3. To the solution from step #2, dibutyl sebacate is added, followed by the addition of CAB-O-SIL® and continued mixing until a homogenous dispersion is formed.
4. The seal coated granules (granules from Example 4) are further coated using a Wurster fluid bed coater with an inlet air temperature of 40°-50° C. and sufficient air volume for fluidization. When the product temperature reaches 30° C., the dispersion from step #3 is sprayed onto the seal coated granules while maintaining the product temperature of 28°-30° C. and sufficient air volume for the fluidization until the target coating weight gain is reached.
5. The coated granules from step #4 are dried.

### Example 7: Functional Coating (FC 1) of Pellets

Seal coated Active Pellets 1 are coated with a functional coating (FC 1) comprising, e.g., KOLLIDON® SR, cellulose acetate, or ETHOCEL™, either alone or at a ratio of [KOLLIDON® SR, cellulose acetate, or ETHOCEL™] to [EUDRAGIT® E PO, PEG, or HPMC] of 95:5.

TABLE 7
Formulation of Functional Coated (FC 1) Pellets

| | Functional Coated | | Functional Coated | |
|---|---|---|---|---|
| | Active Pellets | | Active Pellets | |
| | 1 | | 2 | |
| Components | (% w/w) | mg/dose | (% w/w) | mg/dose |
| Seal coated pellets (pellets | 90.90 | 399.30 | 90.90 | 399.30 |
| from Example 5) | | | | |
| Cellulose acetate or | 7.58 | 33.28 | 7.20* | 31.62 |
| ETHOCEL ™ | | | | |
| EUDRAGIT ® E PO, | NA | NA | 0.38* | 1.66 |
| PEG or HPMC | | | | |
| CAB-O-SIL ® | 0.38 | 1.66 | 0.38 | 1.66 |
| Dibutyl sebacate | 1.14 | 4.99 | 1.14 | 4.99 |
| Solvent system for coating: | | | | |
| Acetone | 90.00 | NA | 90.00 | NA |
| Purified water | 10.00 | NA | 10.00 | NA |
| Total | 100.00 | 439.23 | 100.00 | 439.23 |

*Ratio of cellulose acetate or ETHOCEL ™:EUDRAGIT ® E PO, PEG, or HPMC (95:5)

### Coating Procedure:

1. Cellulose acetate or ETHOCEL™ is added to acetone in a stainless steel container and mixed until a clear solution is formed.
2. To the solution from step #1, purified water is added, [optionally with EUDRAGIT® E PO, PEG, or HPMC (see, e.g., Functional Coated Active Pellets 2, above)], and mixed for ˜5 minutes until a clear solution formed.
3. To the solution from step #3, dibutyl sebacate is added, followed by CAB-O-SIL® and continued mixing until a homogenous dispersion is formed.
4. The seal coated pellets (pellets from Example 5) are further coated using a Wurster fluid bed coater with an inlet air temperature of 40°-50° C. and sufficient air volume for fluidization. When the product temperature reaches 30° C., the dispersion from step #4 is sprayed onto the pellets while maintaining the product temperature of 28°-30° C. and sufficient air volume for the fluidization until the target coating weight gain is reached.
5. The coated pellets from step #4 are dried.

### Example 8: Functional Coating (FC 2) of Granules

Functional coated (FC 1-coated) Active Granules are coated with a secondary functional coat (FC 2) comprising EUDRAGIT® E PO, either alone or at a ratio of [KOLLIDON® SR, cellulose acetate, or ETHOCEL™] to EUDRAGIT® E PO of 60:40.

TABLE 8
Formulation of Functional Coated (FC 2) Granules

| | Functional Coated | | Functional Coated | |
|---|---|---|---|---|
| | Active Granules | | Active Granules | |
| | 1 | | 2 | |
| Components | (% w/w) | mg/dose | (% w/w) | mg/dose |
| FC 1-coated granules | 76.92 | 144.00 | 76.92 | 144.00 |
| (granules from | | | | |
| Example 6) | | | | |
| Cellulose acetate or | 11.54* | 21.60 | NA | NA |
| ETHOCEL ™ | | | | |
| EUDRAGIT ® E PO | 7.69* | 14.4 | 19.23 | 36.00 |
| CAB-O-SIL ® | 0.97 | 1.80 | 0.97 | 1.80 |
| Dibutyl sebacate | 2.88 | 5.40 | 2.88 | 5.40 |
| Solvent system for coating: | | | | |
| Acetone | 90.00 | NA | 90.00 | NA |
| Purified water | 10.00 | NA | 10.00 | NA |
| Total | 100.00 | 187.20 | 100.00 | 187.20 |

*Ratio of cellulose acetate or ETHOCEL ™:EUDRAGIT ® E PO (60:40)

### Coating Procedure:

1. EUDRAGIT® E PO is added to acetone in a stainless steel container and mixed until a clear solution is formed.
2. To the solution from step #1, purified water is added, optionally with cellulose acetate or ETHOCEL™, and mixed for ˜5 minutes until a clear solution is formed.
3. To the solution from step #2, dibutyl sebacate is added, followed by CAB-O-SILR and continued mixing until a homogenous dispersion is formed.
4. FC 1 coated granules (granules from Example 6) are further coated using a Wurster fluid bed coater with an inlet air temperature of 40°-50° C. and sufficient air volume for fluidization. When the product temperature reaches 30° C., the dispersion from step #3 is sprayed onto the granules while maintaining the product temperature of 28°-30° C. and sufficient air volume for the fluidization until the target coating weight gain is reached.
5. The coated granules from step #4 are dried.

### Example 9: Functional Coating (FC 2) of Pellets

Functional coated (FC 1-coated) Active Pellets are coated with a secondary functional coat (FC 2) comprising EUDRAGIT® E PO, either alone or at a ratio of [KOLLIDON® SR, cellulose acetate, or ETHOCEL™] to EUDRAGIT® E PO of 60:40.

TABLE 9
Formulation of Functional Coated (FC 2) Pellets

| | Functional | | Functional | |
|---|---|---|---|---|
| | Coated Active | | Coated Active | |
| | Pellets 1 | | Pellets 2 | |
| Components | (% w/w) | mg/dose | (% w/w) | mg/dose |
| FC 1-coated pellets | 90.90 | 439.23 | 90.90 | 439.23 |
| (pellets from Example 7) | | | | |
| Cellulose acetate or | 4.55* | 21.96 | NA | NA |
| ETHOCEL ™ | | | | |
| EUDRAGIT ® E PO | 3.03* | 14.64 | 7.58 | 36.60 |
| CAB-O-SIL ® | 0.38 | 1.83 | 0.38 | 1.83 |
| Dibutyl sebacate | 1.14 | 5.49 | 1.14 | 5.49 |
| Solvent system for coating | | | | |
| Acetone | 90.00 | NA | 90.00 | NA |
| Purified water | 10.00 | NA | 10.00 | NA |
| Total | 100.00 | 483.15 | 100.00 | 483.15 |

*Ratio of cellulose acetate or ETHOCEL ™:EUDRAGIT ® E PO (60:40)

### Coating Procedure:

1. EUDRAGIT® E PO is added to acetone in a stainless steel container and mixed until a clear solution is formed.
2. To the solution from step #1, water is added, optionally with cellulose acetate or ETHOCEL™, and mixed for ˜5 minutes until a clear solution is formed.
3. To the solution from step #2, dibutyl sebacate is added, followed by CAB-O-SIL® and continued mixing until a homogenous dispersion is formed.
4. FC 1 coated pellets (pellets from Example 7) are further coated using a Wurster fluid bed coater with an inlet air temperature of 40°-50° C. and sufficient air volume for fluidization. When the product temperature reaches 30° C., the dispersion from step #3 is sprayed onto the pellets while maintaining the product temperature of 28°-30° C. and sufficient air volume for the fluidization until the target coating weight gain is reached.
5. The coated pellets from step #4 are dried.

### Example 10: Triggering Granules

Triggering Granules were prepared as described below.

TABLE 10
Formulation of Triggering Granules

| | Triggering Granule 1 | |
|---|---|---|
| Components | (% w/w) | mg/dose |
| Magnesium hydroxide | 50.00 | 175.00 |
| Anhydrous dibasic calcium | 40.28 | 141.00 |
| phosphate | | |
| Crospovidone | 4.29 | 15.00 |
| Hydroxypropyl cellulose | NA | NA |
| Sodium lauryl sulfate | 1.43 | 5.00 |
| Croscarmellose sodium | 2.00 | 7.00 |
| Magnesium stearate | 1.00 | 3.50 |
| Colloidal silicon dioxide | 1.00 | 3.50 |
| Total | 100.00 | 350.00 |

### Manufacturing Procedure:

1. Magnesium hydroxide was added to anhydrous dibasic calcium phosphate (˜50%), crospovidone, hydroxypropyl cellulose, and sodium lauryl sulfate in a high shear granulator and mixed using an impeller and chopper to achieve a uniform blend.
2. The blend from step #1 was granulated by wet granulation.
3. The granules from step #2 were dried at 40° C. using an air oven until the LOD was <1%.
4. Extragranular excipients (anhydrous dibasic calcium phosphate (˜50%), croscarmellose sodium, magnesium stearate, colloidal silicon dioxide) were added to the dried granules and mixed using a V blender to achieve a uniform blend.

### Example 11: Triggering Granules

Triggering Granules are prepared as described below.

TABLE 11
Formulation of Triggering Granules

| | Triggering Granule 2 | | Triggering Granule 3 | | Triggering Granule 4 | |
|---|---|---|---|---|---|---|
| Components | (% w/w) | mg/dose | (% w/w) | mg/dose | (% w/w) | mg/dose |
| Magnesium hydroxide | 58.33 | 175.00 | 50.00 | 175.00 | 58.33 | 175.00 |
| Anhydrous dibasic calcium | 21.67 | 65.00 | NA | NA | NA | NA |
| phosphate | | | | | | |
| Crospovidone | 4.50 | 13.50 | 4.29 | 15.00 | 4.50 | 13.50 |
| Tri-calcium phosphate | NA | NA | 40.28 | 141.00 | 21.67 | 65.00 |
| Hydroxypropyl cellulose | 10.00 | 30.00 | NA | NA | 10.00 | 30.00 |
| Sodium lauryl sulfate | 1.50 | 4.50 | 1.43 | 5.00 | 1.50 | 4.50 |
| Croscarmellose sodium | 2.00 | 6.00 | 2.00 | 7.00 | 2.00 | 6.00 |
| Magnesium stearate | 1.00 | 3.00 | 1.00 | 3.50 | 1.00 | 3.00 |
| Colloidal silicon dioxide | 1.00 | 3.00 | 1.00 | 3.50 | 1.00 | 3.00 |
| Total | 100.00 | 300.00 | 100.00 | 350.00 | 100.00 | 300.00 |

### Manufacturing Procedure:

1. Magnesium hydroxide is added to anhydrous dibasic calcium phosphate (˜50%), crospovidone, hydroxypropyl cellulose, and sodium lauryl sulfate in a high shear granulator and mixed using an impeller and chopper to achieve a uniform blend.
2. The blend from step #1 is granulated by wet granulation.
3. The granules from step #2 are dried at 40° C. using a fluid bed dryer or an air oven until the LOD is <1%.
4. Extragranular excipients (anhydrous dibasic calcium phosphate (˜50%), croscarmellose sodium, magnesium stearate, colloidal silicon dioxide) are added to the dried granules and mixed using a V blender to achieve a uniform blend.

### Example 12: Viscosity Enhancing Granules

Viscosity Enhancing Granules were prepared with a mean particle size of 500 μm.

TABLE 12
Formulation of Viscosity Enhancing Granules

| | Viscosity | | Viscosity | |
|---|---|---|---|---|
| | Enhancing | | Enhancing | |
| | Granules 1 | | Granules 2 | |
| Components | (% w/w) | mg/dose | (% w/w) | mg/dose |
| POLYOX ® | 77.12 | 57.84 | 50.44 | 37.83 |
| WSR coagulant | | | | |
| Crospovidone/ | NA | NA | 28.00 | 21.00 |
| starch 1500 | | | | |
| Hydroxypropyl | 9.41 | 7.06 | 9.41 | 7.06 |
| methylcellulose, | | | | |
| K200M | | | | |
| KOLLIDON ® SR | 4.71 | 3.53 | 4.71 | 3.53 |
| Triethyl citrate | 4.56 | 3.42 | 3.24 | 2.43 |
| Docusate sodium | 2.00 | 1.50 | 2.00 | 1.50 |
| α-dl-Tocopherol | 0.20 | 0.15 | 0.20 | 0.15 |
| Aerosil 200 | 2.00 | 1.50 | 2.00 | 1.50 |
| Total | 100.00 | 75.00 | 100.00 | 75.00 |

### Manufacturing Procedure:

1. POLYOX® WSR coagulant was added to hydroxypropyl methylcellulose K200M, KOLLIDON® SR, docusate sodium, and crospovidone/starch 1500 in a high shear granulator and mixed to achieve a uniform powder mix using impeller and chopper.
2. A solution of α-dl-tocopherol solution and triethyl citrate was sprayed onto the powder mix from step #1 to achieve a uniform blend.
3. Aerosil 200 was added to the blend from step #2 and mixed to achieve a uniform blend using an impeller and chopper.
4. The blend from step #3 was granulated by hot-melt extrusion.
5. The granules from step #4 were processed using cryomilling to a mean particle size of 500 μm.
6. The granules were spheronized using rotor.

### Example 13: Crush-Resistant Active Granules

Active Granules were prepared for use in a 10 mg and 40 mg oxycodone hydrochloride dosage form.

TABLE 13
Formulation of Active Granules

| | Active Granule 5 | | Active Granule 6 | |
|---|---|---|---|---|
| Components | (% w/w) | mg/dose | (% w/w) | mg/dose |
| Oxycodone hydrochloride | 30.00 | 30.00 | NA | NA |
| Hydrocodone bitartrate | NA | NA | 10.00 | 10.00 |
| POLYOX ® WSR coagulant | 50.85 | 50.85 | 70.44 | 70.44 |
| Hydroxypropyl methyl | 9.41 | 9.41 | 9.41 | 9.41 |
| cellulose, K 200M | | | | |
| Kollidon SR | 4.71 | 4.71 | 4.71 | 4.71 |
| Triethyl citrate | 2.83 | 2.83 | 3.24 | 3.24 |
| Docusate sodium | 2.00 | 2.00 | 2.00 | 2.00 |
| α-dl-Tocopherol | 0.20 | 0.20 | 0.20 | 0.20 |
| Total | 100.0 | 100.0 | 100.0 | 100.0 |

### Manufacturing Procedure:

1. Oxycodone hydrochloride (Active Granule 5) or Hydrocodone bitartrate (Active Granule 6), POLYOX® WSR coagulant, hydroxypropyl methylcellulose, K 200M, Kollidon SR, and docusate sodium were added to a high-shear granulator and mixed into a uniform powder mix using an impeller and a chopper at medium speeds.
2. A solution in isopropyl alcohol of α-dl-tocopherol and triethyl citrate was sprayed onto the powder mix from step #1 to achieve a uniform blend.
3. The blend from step #2 was granulated by hot-melt extrusion followed by pelletization.
4. The pellets from step #3 were processed using cryomilling.
5. The pellets from step #4 were seal coated and further coated with a functional coat.

### Example 14: Seal Coating of Granules

Active Granules were coated with a seal coat.

TABLE 14
Formulation of Seal Coated Granules

| | Seal Coated | |
|---|---|---|
| | Granules 5 and 6 | |
| Components | (% w/w) | mg/dose |
| Active Granules | 83.33 | 100.00 |
| (oxycodone hydrochloride/ | | |
| hydrocodone bitartrate | | |
| (Granules 5/6)) | | |
| METHOCEL ™ E5 Premium LV | 14.82 | 17.78 |
| Dibutyl sebacate | 1.48 | 1.78 |
| CAB-O-SIL ® | 0.37 | 0.44 |
| Solvent system for coating: | | |
| Purified water | 30.00 | NA |
| Dehydrated alcohol | 70.00 | NA |
| Total | 100.00 | 120.00 |

### Coating Procedure:

1. METHOCEL™ E5 was added to dehydrated alcohol in a stainless steel container and mixed until dispersed uniformly.
2. To the dispersion from step #1, the purified water was added and mixed until a clear solution was obtained.
3. To the solution from step #2, dibutyl sebacate was added, followed by the addition of CAB-O-SIL® and continued mixing until a homogenous dispersion was obtained.
4. The granules from Example 13 were coated using a Wurster fluid bed coater with an inlet air temperature of 40°-50° C. and sufficient air volume for fluidization. When the product temperature reached 30° C., the dispersion from step #3 was sprayed onto the granules while maintaining the product temperature of 28°-30° C. and sufficient air volume for the fluidization until the target coating weight gain was reached.
5. The coated granules from step #4 were dried.

### Example 15: Functional Coating (FC 1) of Granules

Seal coated oxycodone hydrochloride granules (Granule 5 from Example 14) were coated with a functional coat at a ratio of cellulose acetate to Eudragit® E PO of 95:5, whereas, hydrocodone bitartrate granules (Granule 6 from Example 14) were coated with a functional coat at a cellulose acetate to Eudragit® E PO ratio of 98:2.

TABLE 15
Formulation of functional coated active granules (FC1)

| | FC 1 Coated | | FC 1 Coated | |
|---|---|---|---|---|
| | Granule 5 | | Granule 6 | |
| Composition | (% w/w) | mg/dose | (% w/w) | mg/dose |
| Seal coated | 62.50 | 120.00 | NA | NA |
| oxycodone | | | | |
| hydrochloride | | | | |
| granules (Seal | | | | |
| coated Granule 5) | | | | |
| Seal coated | NA | NA | 62.50 | 120.00 |
| hydrocodone bitartrate | | | | |
| granules (Seal coated | | | | |
| Granule 6) | | | | |
| Cellulose Acetate | 29.69 | 57.00 | 31.98 | 61.40 |
| Eudragit ® E PO | 1.56 | 3.00 | 0.68 | 1.30 |
| Dibutyl sebacate | 4.69 | 9.00 | 3.23 | 6.20 |
| Colloidal silicon | 1.56 | 3.00 | 1.61 | 3.10 |
| dioxide | | | | |
| Solvent system | | | | |
| for coating | | | | |
| Acetone* | 90.00 | NA | 90.00 | NA |
| Purified water* | 10.00 | NA | 10.00 | NA |
| Total | 100.00 | 192.00 | 100.00 | 192.00 |

*Removed during process

### Coating Procedure:

1. EUDRAGIT® E PO was added to acetone in a stainless steel container and mixed until a clear solution was obtained.
2. To the solution from step #1 cellulose acetate was added and mixed until a clear solution was obtained.
3. The purified water was added to the solution from step #2 and mixed for ˜5 minutes.
4. To the solution from step #3 dibutyl sebacate was added followed by colloidal silicon dioxide and continued mixing until a homogenous dispersion was obtained.
5. The seal coated granules from Example 14 were further coated with the dispersion from step #4 using a Wurster fluid bed coater with an inlet air temperature of 40°-50° C. and sufficient air volume for fluidization. When the product temperature reached 30° C., the dispersion from step #4 was sprayed onto the seal coated granules while maintaining the product temperature of 28°-30° C. and sufficient air volume for the fluidization until the target coating weight gain was reached.
6. The coated granules from step #5 were dried.

### Example 16: Functional Coating (FC 2) of Granules

Functional coated active granules (FC1) were further coated with Eudragit® E PO (FC2).

TABLE 16
Formulation of functional coated active granules (FC2)

| | FC 2 Coated | | FC 2 Coated | |
|---|---|---|---|---|
| | Granule 5 | | Granule 6 | |
| Composition | (% w/w) | mg/dose | (% w/w) | mg/dose |
| Functional coated | 62.50 | 192.00 | NA | NA |
| oxycodone hydro- | | | | |
| chloride granules | | | | |
| (FC1 coated Granule 5) | | | | |
| Functional coated | NA | NA | 62.50 | 192.00 |
| hydrocodone | | | | |
| bitartrate granules | | | | |
| (FC1 coated Granule 6) | | | | |
| Eudragit ® E PO | 31.25 | 96.00 | 26.79 | 82.29 |
| Polyethylene glycol | 3.13 | 9.60 | 2.68 | 8.23 |
| Colloidal silicon | 3.13 | 9.60 | 4.02 | 12.34 |
| dioxide | | | | |
| Talc | NA | NA | 4.02 | 12.34 |
| Solvent system | | | | |
| for coating | | | | |
| Acetone* | 40.00 | NA | 40.00 | NA |
| Isopropyl alcohol* | 60.00 | NA | 60.00 | NA |
| Total | 100.01 | 307.20 | 100.01 | 307.20 |

*Removed during process

### Coating Procedure:

1. Eudragit® E PO was added to a mixture of acetone and isopropyl alcohol in a stainless steel container and mixed until a clear solution was obtained.
2. To the solution from step #1 polyethylene glycol was added followed by colloidal silicon dioxide and/or talc and continued mixing until a homogenous dispersion was formed.
3. The functional coated granules (FC1) from Example 15 were further coated using a Wurster fluid bed coater with an inlet air temperature of 40°-50° C. and sufficient air volume for fluidization. When the product temperature reached 30° C., the dispersion from step #2 was sprayed onto the functional coated granules (FC1) while maintaining the product temperature of 28°-30° C. and sufficient air volume for the fluidization until the target coating weight gain was reached.
4. The coated granules from step #3 were dried.

### Example 17: Over Coating of Active Granules

Functional coated active granules (FC2) were finally coated with an over coat.

TABLE 17
Formulation of over coated active granules

| | Over Coated | | Over Coated | |
|---|---|---|---|---|
| | Granules 5 | | Granules 6 | |
| Composition | (% w/w) | mg/dose | (% w/w) | mg/dose |
| Functional coated | 86.96 | 307.20 | NA | NA |
| oxycodone hydro- | | | | |
| chloride granules | | | | |
| (FC2 coated | | | | |
| Granule 5) | | | | |
| Functional coated | NA | NA | 83.33 | 307.20 |
| hydrocodone bitartrate | | | | |
| granules (FC2 coated | | | | |
| Granule 6) | | | | |
| Methocel E5 | 10.03 | 35.45 | 14.82 | 54.62 |
| Premium LV | | | | |
| Triethyl citrate | 1.00 | 3.54 | 1.48 | 5.46 |
| Talc | 2.01 | 7.09 | 0.37 | 1.36 |
| Solvent system | | | | |
| for coating | | | | |
| Purified water* | 20.00 | NA | 20.00 | NA |
| Dehydrated alcohol* | 80.00 | NA | 80.00 | NA |
| Total | 100.00 | 353.28 | 100.00 | 368.64 |

*Removed during process

### Coating Procedure:

1. Methocel E5 was added to dehydrated alcohol in a stainless steel container and mixed until it dispersed uniformly.
2. To the dispersion from step #1 the purified water was added and mixed to obtain a clear solution.
3. To the solution from step #2 triethyl citrate was added followed by the addition of talc and continued mixing until a homogenous dispersion was obtained.
4. The functional coated granules from Example 16 were coated with the dispersion from step #3 using a Wurster fluid bed coater with an inlet air temperature of 40°-50° C. and sufficient air volume for fluidization. When the product temperature reached 30° C., the dispersion from step #3 was sprayed onto the granules while maintaining the product temperature of 28°-30° C. and sufficient air volume for the fluidization until the target coating weight gain was reached.
5. The coated granules from step #4 were dried.

### Example 18: Viscosity Enhancing Granules

TABLE 18
Composition of Viscosity Enhancing Granules

| | Viscosity Enhancing | |
|---|---|---|
| | Granules | |
| Composition | (% w/w) | mg/dose |
| Polyox ® WSR coagulant | 42.03 | 31.52 |
| Crospovidone | 23.33 | 17.50 |
| Hydroxypropyl methylcellulose, K200M | 7.83 | 5.87 |
| Kollidon ® SR | 3.93 | 2.95 |
| Triethyl citrate | 2.70 | 2.03 |
| Docusate sodium | 1.67 | 1.25 |
| α-dl-Tocopherol | 0.17 | 0.13 |
| Colloidal silicon dioxide | 1.67 | 1.25 |
| Seal coating | | |
| Methocel E5 Premium LV | 14.82 | 11.12 |
| Triethyl citrate | 1.48 | 1.11 |
| Colloidal silicon dioxide | 0.37 | 0.28 |
| Solvent system for coating | | |
| Purified water* | 20.00 | NA |
| Dehydrated alcohol* | 80.00 | NA |
| Total | 100.00 | 75.00 |

*Removed during process

a. Manufacturing Procedure:
1. Polyox® WSR coagulant was added to hydroxypropyl methylcellulose K200M, Kollidon® SR, docusate sodium and crospovidone in a high shear granulator and mixed to achieve a uniform powder mix using impeller and chopper.
2. A solution of α-dl-tocopherol solution and triethyl citrate was sprayed onto the powder mix from step #1 to achieve a uniform blend.
3. Colloidal silicon dioxide was added to the blend from step #2 and mixed to achieve a uniform blend using an impeller and chopper.
4. The blend from step #3 was subjected to hot-melt extrusion followed by pelletization.
5. The pellets from step #4 were subjected to size reduction process using cryomilling.
6. The granules from step #5 were seal coated.
   b. Seal Coating Procedure:
1. Methocel E5 was added to dehydrated alcohol in a stainless steel container and mixed until it dispersed uniformly.
2. To the dispersion from step #1 the purified water was added and mixed until a clear solution was obtained.
3. To the solution from step #2 triethyl citrate was added followed by the addition of colloidal silicon dioxide and continued mixing until a homogenous dispersion was obtained.
4. The granules from “step a” were coated using a Wurster fluid bed coater with an inlet air temperature of 40°-50° C. and sufficient air volume for fluidization. When the product temperature reached 30° C., the dispersion from step #3 was sprayed onto the viscosity enhancing granules while maintaining the product temperature of 28°-30° C. and sufficient air volume for the fluidization until the target coating weight gain was reached.
5. The coated granules from step #4 were dried.

### Example 19: Triggering Granules

TABLE 19
Formulation of Triggering Granules

| | Triggering Granules | |
|---|---|---|
| Composition | (% w/w) | mg/dose |
| Magnesium hydroxide | 82.02 | 135.00 |
| Mannitol | 13.88 | 22.85 |
| Crospovidone | 4.10 | 6.75 |
| Purified water | NA | NA |
| Total | 100.00 | 164.60 |

### Manufacturing Procedure:

1. Magnesium hydroxide was added to mannitol and crospovidone in a high shear granulator and mixed using an impeller and chopper to obtain a uniform blend.
2. The blend from step #1 was granulated using purified water.
3. The granules from step #2 were dried at 40° C. using a forced air oven until the LOD was <1%.

### Example 20: Formulation Development

The final dosage form was developed as a tablet using Active Granules, Triggering Granules, and optionally, with Viscosity Enhancing Granules, and other excipients.

TABLE 20
Tablet composition of Oxycodone hydrochloride ER tablets, 40 mg
(Tablet 5) and Hydrocodone bitartrate ER tablets, 20 mg (Tablet 6)

| | Tablet 5 | Tablet 6 |
|---|---|---|
| Tablet Composition | mg/unit | mg/unit |
| Over coated oxycodone hydrochloride | 417.04* | NA |
| granules (Granules 5 from Example 17) | | |
| Over coated hydrocodone bitartrate | NA | 568.00** |
| granules (Granules 6 from Example 17) | | |
| Viscosity enhancing granules (Granules | 75.00 | NA |
| from Example 18) | | |
| Triggering granules (Granules from | 164.60 | 164.60 |
| Example 19) | | |
| Microcrystalline cellulose | 211.30 | 425.40 |
| Mannitol | 30.00 | 10.00 |
| Hydroxypropyl cellulose | 8.00 | 8.00 |
| Croscarmellose Sodium | 20.00 | 20.00 |
| Magnesium Stearate | 4.00 | 4.00 |
| Tablet weight | 983.94 | 1200.00 |

*Equivalent weight of over coated oxycodone hydrochloride granules for 40 mg of active
**Equivalent weight of over coated hydrocodone bitartrate granules for 20 mg of active

### Manufacturing Procedure:

1. A uniform blend of over coated active granules (Granules 5 & 6 from example 17), viscosity enhancing granules (Granules from Example 18), triggering granules (Granules from Example 19), microcrystalline cellulose, mannitol, hydroxypropyl cellulose and croscarmellose sodium was made using a V-blender.
2. To the blend from step #1, magnesium stearate was added and blended for 3 minutes using a V-blender.
3. The blend from step #2 was compressed into tablets using a tablet press.

The present disclosure is well adapted to attain the ends and advantages mentioned, as well as those that are inherent therein. The particular embodiments disclosed above are illustrative only, as the present disclosure can be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above can be altered or modified, and all such variations, including but not limited to substitution of different opioid active agents, are considered within the scope and spirit of the present disclosure. Various publications, patents, and patent application are cited herein, the contents of which are hereby incorporated by reference herein in their entireties.

1-57. (canceled)

58. A solid, oral, multi-particulate dosage form, comprising abuse deterrent and overdose protection characteristics, comprising:
(a) a first population of particulates comprising a therapeutically effective amount an opioid embedded in a polymer matrix, a primary functional coat layer (FC 1) over the polymer matrix, a secondary functional coat layer (FC 2) over FC 1, and an over coat over FC 2,
wherein FC 1 comprises a nonionic water-insoluble polymer and a cationic polymer, FC 2 comprises a cationic polymer, and the over coat comprises a nonionic water-soluble polymer; and
(b) a second population of particulates comprising an alkaline agent,
wherein the dosage form is suitable for twice-daily administration and provides an extended release of the opioid for a period of at least about 4 hours, and releases less than about 40% of the opioid from the dosage form at about 1 hour; and
wherein, when two or more dosage units are consumed, the alkaline agent raises the gastric pH to a value of greater than about 5 to further extend the release of the opioid from the dosage form.

59. A solid, oral, multi-particulate dosage form, comprising abuse deterrent and overdose protection characteristics, comprising:
(a) a first population of particulates comprising a therapeutically effective amount of an opioid embedded in a polymer matrix, a primary functional coat layer (FC 1) over the polymer matrix, a secondary functional coat layer (FC 2) over FC 1, and an over coat over FC 2,
wherein FC 1 comprises a nonionic water-insoluble polymer and a cationic polymer, FC 2 comprises a cationic polymer, and the over coat comprises a nonionic water-soluble polymer; and
(b) a second population of particulates comprising an alkaline agent,
wherein the dosage form is suitable for once-daily administration and provides an extended release of the opioid for a period of at least about 8 hours, and releases less than about 40% of the opioid from the dosage form at about 1 hour; and
wherein, when two or more dosage units are consumed, the alkaline agent raises the gastric pH to a value of greater than about 5 to further extend the release of the opioid from the dosage form.

60. The dosage form of claim 58, wherein the cationic polymer acts as a pore former in FC 1 at a wt % ratio of nonionic water-insoluble polymer to cationic polymer from about 80:20 to about 99.9:0.1.

61. The dosage form of claim 58, wherein wt % ratio of nonionic water-insoluble polymer to cationic polymer in FC 1 is about 95:5.

62. The dosage form of claim 58, wherein the nonionic water-insoluble polymer present in FC 1 is selected from the group consisting of cellulose acetate, cellulose acetate-based polymers, ethylcellulose, and polyvinyl acetate polymers.

63. The dosage form of claim 62, wherein the nonionic water-insoluble polymer is cellulose acetate.

64. The dosage form of claim 58, wherein FC 1 further comprises a nonionic water-soluble polymer selected from the group consisting of hydroxypropyl methylcellulose (HPMC) and polyethylene glycol (PEG).

65. The dosage form of claim 58, wherein the cationic polymer present in FC 1 and FC 2, is a copolymer based on dimethylaminoethyl methacrylate, butyl methacrylate, and methyl methacrylate.

66. The dosage form of claim 58, wherein the polymer matrix of the first population of particulates further comprises a nonionic polymer and/or an anionic polymer.

67. The dosage form of claim 66, wherein the anionic polymer is a carbomer.

68. The dosage form of claim 66, wherein the nonionic polymer comprises a copolymer of ethyl acrylate, methyl methacrylate, and a low content of methacrylic acid ester with quaternary ammonium groups (ammonium methacrylate copolymer), hydroxypropyl cellulose, HPMC, hydroxyethylcellulose, ethylcellulose, cellulose acetate butyrate, cellulose acetate, polyvinyl acetate polymers, or polyethylene oxide polymers.

69. The dosage form of claim 68, wherein the nonionic polymer is a mixture of a polyethylene oxide polymer and HPMC.

70. The dosage form of claim 58, wherein the alkaline agent is selected from the group consisting of aluminum hydroxide, sodium hydroxide, potassium hydroxide, calcium hydroxide, magnesium hydroxide, calcium carbonate, sodium carbonate, potassium bicarbonate, sodium bicarbonate, ammonia, tertiary sodium phosphate, diethanolamine, ethylenediamine, N-methylglucamine, L-lysine, and combinations thereof.

71. The dosage form of claim 70, wherein the alkaline agent is magnesium hydroxide.

72. The dosage form of claim 58, wherein the first population of particulates further comprises a seal coat disposed between the polymer matrix and FC 1.

73. The dosage form of claim 72, wherein the seal coat comprises a nonionic water-soluble polymer.

74. The dosage form of claim 73, wherein the nonionic water-soluble polymer is HPMC.

75. The dosage form of claim 58, wherein the over coat is the outermost coat on the first population of particulates.

76. The dosage form of claim 58, wherein the first population of particulates further comprises at least one of an additional coating layer between the seal coat and FC1, or between FC 1 and FC 2, or between FC 2 and the over coat.

77. The dosage form of claim 58 wherein the opioid is selected from the group consisting of oxycodone, oxymorphone, hydromorphone, hydrocodone, and pharmaceutically acceptable salts thereof.

78. The dosage form of claim 77, wherein the opioid is oxycodone or a pharmaceutically acceptable salt thereof.

79. The dosage form of claim 58, further comprising a third population of particulates comprising a viscosity-building polymer.

80. The dosage form of claim 79, wherein the viscosity-building polymer is a nonionic polymer and/or an anionic polymer.

81. The dosage form of claim 80, wherein the nonionic polymer is a polyethylene oxide polymer and the anionic polymer is a carbomer.

82. The dosage form of claim 58, wherein the abuse deterrent characteristics comprise syringeability resistance, extractability resistance in aqueous and/or hydro-organic solvents, resistance to alcohol dose dumping, and heat stability of the dosage form; and wherein the heat stability comprises maintaining the abuse deterrent characteristics of the dosage form after exposure to heat at about 100° C., for at least about 2 hours, in an oven; or at about 1200 W, for at least about 13-15 minutes, in a microwave.

83. The dosage form of claim 58, wherein the abuse deterrent characteristics comprise resistance to crushing and grinding of the first population of particulates.

84. The dosage form of claim 58, wherein the abuse deterrent characteristics comprise resistance to segregation of the opioid into a fines fraction of the dosage form upon grinding, wherein the fines fraction comprises a fraction of particulates with a size that can be snorted or insufflated.

85. A method of preparing a multi-particulate dosage form, comprising abuse deterrent and overdose protection characteristics, comprising:
(a) preparing a first population of particulates comprising a therapeutically effective amount of an opioid embedded in a polymer matrix, a primary functional coat layer (FC 1) over the polymer matrix, a secondary functional coat layer (FC 2) over FC 1, and an over coat over FC 2,
wherein FC 1 comprises a nonionic water-insoluble polymer and a cationic polymer, FC 2 comprises a cationic polymer, and the over coat comprises a nonionic water-soluble polymer;
(b) preparing a second population of particulates comprising an alkaline; and
(c) combining the first and second populations of particulates.

86. The method of claim 85, wherein the cationic polymer acts as a pore former in FC 1 at a wt % ratio of nonionic water-insoluble polymer to cationic polymer from about 80:20 to about 99.9:0.1.

87. The dosage form of claim 85, wherein the nonionic water-insoluble polymer present in FC 1 is cellulose acetate.

88. The dosage form of claim 85, wherein the cationic polymer present in FC 1 and FC 2, is a copolymer based on dimethylaminoethyl methacrylate, butyl methacrylate, and methyl methacrylate.

89. The method of claim 85, further comprising combining the particulate populations in either a tablet, a tablet-in-tablet, a bilayer tablet, or a capsule dosage form.

90. A method for providing overdose protection from an opioid overdose, the method comprising orally administering to a subject in need of an opioid, a solid, oral, multi-particulate dosage form comprising:
(a) a first population of particulates comprising a therapeutically effective amount of an opioid or a pharmaceutically acceptable salt thereof embedded in a polymer matrix, a primary functional coat layer (FC 1) over the polymer matrix, a secondary functional coat layer (FC 2) over FC 1, and an over coat over FC 2,
wherein FC 1 comprises a nonionic water-insoluble polymer and a cationic polymer, FC 2 comprises a cationic polymer, and the over coat comprises a nonionic water-soluble polymer; and
(b) a second population of particulates comprising an alkaline agent,
wherein the dosage form provides an extended release of the opioid or pharmaceutically acceptable salt thereof for a period of at least about 4 hours, and releases less than about 40% of the opioid or pharmaceutically acceptable salt thereof from the dosage form at about 1 hour; and
wherein, when two or more dosage units are consumed, the alkaline agent raises the gastric pH to a value of greater than about 5 to further extend the release of the opioid or pharmaceutically acceptable salt thereof from the dosage form.

91. A method for providing analgesia to a subject comprising administering to the subject a solid, oral, multi-particulate opioid dosage form in an extended release overdose protection formulation without impeding release of the opioid when taken as directed, wherein the dosage form comprises:
(a) a first population of particulates comprising a therapeutically effective amount of an opioid or a pharmaceutically acceptable salt thereof embedded in a polymer matrix, a primary functional coat layer (FC 1) over the polymer matrix, a secondary functional coat layer (FC 2) over FC 1, and an over coat over FC 2,
wherein FC 1 comprises a nonionic water-insoluble polymer and a cationic polymer, FC 2 comprises a cationic polymer, and the over coat comprises a nonionic water-soluble polymer; and
(b) a second population of particulates comprising an alkaline agent,
wherein the dosage form provides an extended release of the opioid or pharmaceutically acceptable salt thereof for a period of at least about 4 hours, and releases less than about 40% of the opioid or pharmaceutically acceptable salt thereof from the dosage form at about 1 hour; and
wherein, when two or more dosage units are consumed, the alkaline agent raises the gastric pH to a value of greater than about 5 to further extend the release of the opioid or pharmaceutically acceptable salt thereof from the dosage form.
